(19) United States
(12) Patent Application Publication
(10) Pub. No.: US 2004/0057969 A1
(43) Pub. Date: Mar. 25, 2004

(54) Compositions containing stabilized hepatitis antigen and methods of their use

(76) Inventors: Mark L. Smith, Davis, CA (US); Michael L. Shuler, Ithaca, NY (US)

Correspondence Address: Michael L. Goldman, NIXON PEABODY LLP, Clinton Square, P.O. Box 31051, Rochester, NY, 14603-1051, US

(21) Appl. No.: 10/251,167

(22) Filed: Sep. 20, 2002

(51) Int. Cl. C12Q 1/70; C12Q 1/68; A61K 39/29

(52) U.S. Cl. 424/225.1; 424/227.1; 435/6; 435/5

(57) ABSTRACT

The present invention relates to a composition, which includes a hepatitis B surface antigen stabilized with a milk protein and/or a milk protein component. This composition can be used in an oral vaccine for treatment of hepatitis B. The present invention further relates to methods of immunizing a subject against hepatitis, methods of administrating the composition of the present invention, and methods of producing a stabilized hepatitis B surface antigen protein.

This invention was developed with government funding under National Science Foundation Grant Nos. BES97-08250 and BES0109936. The U.S. Government may have certain rights.

### FIELD OF THE INVENTION

The present invention relates to a composition of a hepatitis B surface antigen which may be stabilized with a milk protein and/or a milk protein component thereof.

### BACKGROUND OF THE INVENTION

Plants and plant cell systems are rapidly becoming established as an efficient platform for the production of pharmaceutically important proteins. Whole plant expression systems provide rapid production scale-up through increased acreage. Plant cell suspension cultures can be grown in inexpensive reactor configurations and should be economically viable in cases where small to moderate quantities of a specific protein are required.

Methods for producing transgenic plants are well known. In a typical transformation scheme, a plant cell is transformed with a DNA construct, in which a “foreign” DNA molecule that is to be expressed in the plant cell is operably linked to a DNA promoter molecule, which will direct expression of the foreign DNA in the host cell, and to a 3′ regulatory region of DNA that will allow proper processing of the RNA transcribed from the target DNA. The choice of foreign DNA to be expressed will be based on the trait, or effect, desired for the transformed plant. The promoter molecule is selected so that the foreign DNA is expressed in the desired plant. Promoters are regulatory sequences that determine the time and place of gene expression. Transcription of DNA is dependent upon the presence of a promoter which is a DNA sequence that directs the binding of RNA polymerase and thereby promotes mRNA synthesis.

Two major advantages of plant systems is that plant viruses are non-pathogenic to humans, and plant DNA contains no elements known to cause cancer. This reduces downstream processing costs, compared to traditional mammalian cell production systems. In addition, plant culture medium formulations are simple, inexpensive, defined and serum free, further reducing costs and eliminating the possibility of prion contamination. Plants have also been shown to possess the capability to perform much of the post-translational processing of mammalian cells, yielding biologically active products.

Efforts have been made by researchers to improve yield, facilitate production and/or improve the safety of protein based vaccines, protein antigens, which are sometimes expressed by host cells of the same (i.e., homologous) species or of a different (i.e., heterologous) species that is safe to handle and/or allow high expression levels in different types of biological systems, such as in plant systems. This is significant as the administration of vaccines to humans and animals aid in the induction of their immune systems to produce antibodies against viruses, bacteria, and other types of pathogenic organisms.

Heterologous hosts used for the expression of immunogenic proteins, include yeast, bacteria, and mammalian cell lines. An important example is exemplified by studies pertaining to the hepatitis B surface antigen (“HBsAg”), previously obtained from plasma of individuals, which now may be expressed in such heterologous hosts. A search for a vaccine against Hepatitis B is significant as it is a widespread and potentially fatal viral disease.

Specifically, HBsAg is a 226 amino acid protein, with a molecular weight of approximately 25 kDa. More particularly, the human plasma-derived HBsAg is recoverable as a 22 nm particle containing both a glycosylated polypeptide (reported as having a molecular weight of 27,000-28,000 daltons) and a nonglycosylated polypeptide (reported as having a molecular weight of 23,000-26,000 daltons). This polypeptide is coded for by the HBsAg gene. Human plasma-derived HBsAg contains cholesterol, is free of phosphatidylinositol and is stable at pH=2 in the presence of pepsin at pH=2.

Moreover, HBsAg is a membrane bound protein with 4 transmembrane regions. In its native form, the HBsAg protein decorates the surface of 22 nm Virus Like Particles (VLPs). These VLPs are composed of approximately 75% protein, 25% lipid, with extensive disulfide bridging between the monomer subunits to generate oligomers of 14-16 units. Several different subtypes of HBsAg exist; however, they all possess the group specific a determinant. Covalent disulfide bonds exist between essentially all monomeric protein molecules of human plasma-derived HBsAg.

In light of the foregoing, any vaccine against the hepatitis B virus must display the correctly folded a determinant as antibodies against this epitope are required to generate an effective immune response. In particular, the immunogenicity of human HBsAg and the ability of the human HBsAg to be used as a vaccine is known to be critically dependent on the disulfide bonded form of the antigen (Mishiro, et al., “A 49,000-Dalton Polypeptide Bearing All Antigenic Determinants and Full Immunogenicity of 22-nm Hepatitis B Surface Antigen Particles,” J. Immunol. 124:1589-1593 (1980)). If few disulfide bonds of HBsAg are present, the immunogenicity of the HBsAg is drastically reduced.

In fact, vaccines used to immunize susceptible persons against infection by hepatitis B virus derived from different biological systems are available. For example, one vaccine is derived by purification of the spherical 22 nm HBsAg from the plasma of humans, infected by hepatitis B virus, who have become chronic producers of surface antigen. However, in order to insure the safety of the vaccine derived from human plasma, multiple steps are required in the purification process in order to inactivate potential contaminating infectious agents.

Although this human derived vaccine has been useful, investigators have sought alternate sources for those immunogenic 22 nm particles. Alternate sources have involved the synthesis or manufacture of HBsAg in different cells, such as mammalian cells and a variety of yeasts, including Saccharomyces cerevisiae (McAleer, et al., “Human Hepatitis B Vaccine From Recombinant Yeast,” 307:178-180 Nature (1984); Petre, et al., “Development of a Hepatitis B Vaccine From Transformed Yeast Cells,” Postgrad. Med. J. 63:73-81 (1987)) or Pichia pastoris (Hardy, et al., “Large-Scale Production of Recombinant Hepatitis B Surface Antigen From Pichia pastoris,” J. Biotechnol. 77:157-167 (2000); Cregg, et al., “High-Level Expression and Efficient Assembly of Hepatitis B Surface Antigen in The Methylotrophic Yeast, Pichia pastoris,” Bio/Technology 5:479-485 (1987)) using conventional molecular cloning techniques. Successful expression of HBsAg in plants has been observed (Kong, et al., “Oral Immunization With Hepatitis B Surface Antigen Expressed In Transgenic Plants,” Proc. Natl. Acad. Sci. USA 98:11539-11544 (2001); Mason, et al., “Expression Of Hepatitis B Surface Antigen In Transgenic Plants,” Proc. Natl. Acad. Sci. USA 89:11745-11749 (1992); and Richter et al., “Production Of Hepatitis B Surface Antigen In Transgenic Plants For Oral Immunization,” Nat Biotechnol. 18:1167-(2000)). In some instances limited success has been achieved in expression of the cloned gene. For example, the 22 nm HBsAg is difficult to make in some prokaryotic organisms, e.g. Escherichia coli. The synthesis of HBsAg in Saccharomyces cerevisiae has been reported by Valenzuela et al., Nature, 298: 347-350 (1982); Hitzeman et al., Nucleic Acid Research, 11: 2745-2763 (1983); and Miyanohara et al., Proc. Nat'l Acad. Sci. USA, 80:1-5 (1983).

With advances in research, yeast and mammalian derived vaccines have become available worldwide and are as efficacious as the classical serum-derived vaccine. Both expression systems which are transformed with a plasmid containing the HBsAg-encoding gene yield 22 nm HBsAg particles that are identical to those excreted by the native virus. As previously indicated, the advantages achieved from such processes relate to safety concerns, consistent quality of materials that are produced, and consist high yields.

Moreover, manufacture of HBsAg in different yeasts, such as Saccharomyces cerevisiae and Pichia pastoris, which are GRAS organisms (i.e., Generally Recognized As Safe substances by the U.S. Food and Drug Administration, with the latter being used for single cell protein production), has lead researchers to believe that use of such organisms and corresponding materials could provide an “edible” vaccine source. Because of simplicity of delivery of vaccines by oral delivery, there is great current interest in discovering new oral vaccine technology. Appropriately delivered, oral immunogens can stimulate both humoral and cellular immunity and have the potential to provide cost-effective, safe vaccines for use in developing countries or inner cities where large-scale parenteral immunization is not practical or extremely difficult to implement. However, crude extracts of S. cerevisiae expressing HBsAg were shown to be poorly immunogenic upon injection into mice as discussed in the U.S. Pat. No. 4,707,542 1987 to Friedman et al.

The concept of edible plant-based vaccines was introduced in 1992, with the expression of the Hepatitis B surface Antigen (HBsAg) in tobacco. (Mason, et al., “Expression of Hepatitis B Surface Antigen in Transgenic Plants,” Proc. Natl. Acad. Sci. USA 89:11745-11749 (1992)).

Potato was later chosen as a model system, and feeding raw tuber expressing the B-subunit of the Escherichia coli heat-labile enterotoxin (LT-B) was shown to protect mice from subsequent challenge with the LT-holotoxin. (Mason, et al., “Edible Vaccine Protects Mice Against Escherichia coli Heat-Labile Enterotoxin (LT): Potatoes Expressing a Synthetic LT-B Gene,” Vaccine 16:1336-1343 (1998); Haq, et al., “Oral Immunization With a Recombinant Bacterial Antigen Produced in Transgenic Plants,” Science 268:714-716 (1995)). Clinical trials, again feeding raw tubers expressing this potent oral immunogen, provided proof of principle in humans. (Tacket, et al., “Immunogenicity in Humans of a Recombinant Bacterial Antigen Delivered in a Transgenic Potato,” Nat. Med. 4:607-609 (1998)). More recently, pre-clinical animal feeding trials with potato tubers expressing HBsAg successfully elicited a primary serum immune response demonstrating that a plant-produced antigen, from a non-enteric human pathogen (hepatitis B virus), was orally immunogenic. (Richter, et al., “Production of hepatitis B Surface Antigen in Transgenic Plants for Oral Immunization,” Nat. Biotechnol 18:1167-1171 (2000)). Furthermore, mice injected with a partially purified preparation of tobacco-derived HbsAg produced a favorable immune response which was qualitatively similar to that obtained by immunization with the commercial vaccine.

However, these initial studies all employed the plant material “as is” (i.e., without additional processing) such that differences in expression due to plant development, environmental conditions etc. make it difficult to ensure consistency of dose and potency. (Stein, et al., “The Regulation of Biologic Products Derived From Bioengineered Plants,” Curr. Opin. Biotech. 12:308-311 (2001)). Therefore, a certain degree of processing of the plant material will be required. For a minimally processed product, microbiological considerations ensue. Since no known plant virus is pathogenic to humans (Stein, et al., “The Regulation of Biologic Products Derived From Bioengineered Plants,” Curr. Opin. Biotech. 12:308-311 (2001)), these are of minimal concern. Other bioburdens merit greater attention, e.g. toxigenic soil bacteria and fungi. (Miele, L., “Plants as Bioreactors for Biopharmaceuticals,” Trends Biotechnol 15:45-50 (1997)). Here, the efficient and cost-effective procedures developed in the food industry are of value. For example, lye peelers, which employ 5-15% sodium hydroxide solutions and elevated temperatures (82-105° C.), effectively remove potato skin and would inactivate soil-borne pathogens on the tuber surface. (Gould, W. A., Potato Production, Processing & Technology, Timonium, Md.: CTI Publications, p 78 (1999)).

Another factor, which may affect the successful use of crude host extracts, such as plant extracts expressing HBsAg, relates to the quantitation of the antigen products in crude plant extracts. It is important to note that with limited processing such an antigen resides in a complex background of plant proteins and can exist in various forms, due to different extents of processing, complicating this determination. This is particularly true for HBsAg which undergoes a complex sequence of post-translational modifications as follows: insertion into the ER membrane, disulfide bond-mediated dimerization followed by budding into the ER lumen, and oligomerization. (Patzer, et al., “Intracellular Assembly and Packaging of Hepatitis B Surface Antigen Particles Occur in the Endoplasmic Reticulum,” J. Virol. 58:884-892 (1986); Huovila, et al., “Hepatitis B Surface Antigen Assembles in a Post-ER Pre-Golgi Compartment,” J. Cell Biol. 118:1305-1320 (1992)). For highly purified injectable preparations, determining the total antigen dose is relatively straightforward; a total protein assay provides an accurate value.

Antigen stability in the crude plant extract is also a concern. As a result of processing of the plant material, intracellular compartmentalization will be compromised (Loomis, W. D., “Overcoming Problems of Phenolics and Quinones in the Isolation of Plant Enzymes and Organelles,” Meth. Enzymol. 31:528-544 (1994)) exposing the antigen to proteases, polyphenol oxidases, and plant phenolics which could impair the immunogenic epitopes of HBsAg.

Another consideration relates to the cost of processing such materials. For a typical fermentation-produced biologic, the major manufacturing cost is often downstream processing, in particular protein purification. (Wheelwright, S. M. “Designing Downstream Processes for Large Scale Protein Purification,” Bio/Technology 5:789 (1987)). Minimally processed antigen preparations are expected to reduce this cost significantly, provided accurate quantitation and antigen stability can be achieved.

The present invention is directed to overcoming the deficiencies in the prior art.

### SUMMARY OF THE INVENTION

The present invention relates to a composition, which includes a stabilized hepatitis B surface antigen which can contain a milk protein and/or a milk protein component.

The present invention relates to an oral vaccine for treatment of hepatitis B, which includes a composition formed from a stabilized hepatitis B surface antigen stabilized which can contain a milk protein and/or a milk protein component and a carrier.

The present invention relates to a method of immunizing a subject against hepatitis, which comprises administering to the subject a stabilized composition of hepatitis B surface antigen which can contain a milk protein and/or a milk protein component.

The present invention relates to a method of producing a stabilized hepatitis B surface antigen protein, which comprises providing a cell culture suspension containing hepatitis B surface antigen and extracting the hepatitis B surface antigen with a buffer that can contain a milk protein and/or a milk protein component to yield a stabilized hepatitis B surface antigen protein extract.

The present invention also relates to a method of increasing immunogenicity of a hepatitis B surface antigen. This involves providing a cell culture medium comprising a hepatitis B surface antigen and extracting the hepatitis B surface antigen with a buffer containing a pH of 7 to 12 to yield an extract. The extract is stored at 0 to 10° C. so that the hepatitis B surface antigen has an increased immunogenicity.

An advantage of the present invention is that milk proteins or a corresponding protein component thereof have a stabilizing effect on HBsAg particles. For example, addition of milk protein or a corresponding protein components could potentially be used to stabilize plant-derived HBsAg, for its incorporation into an oral vaccine delivery system. Such a use of milk protein sources, e.g., such as soy protein, are advantageous, resulting in improvement in HBsAg stability due to such factors as an increase in solution protein concentration. No work has been performed with soy milk or soy protein.

Methods are described for increasing the fraction of plant-derived antigen which expresses the immunologically relevant a-determinant specific epitopes.

Several factors and methods for stabilizing the plant-derived HBsAg antigen are disclosed, including the optimum detergent to tissue ratio, the presence of sodium ascorbate during extraction, and the use of skim milk or its protein components in the extraction buffer.

Extraction conditions (detergent to plant material ratio) required to generate a relatively uniform population of HBsAg VLPs from the initial complex intracellular form of plant-derived HBsAg are disclosed.

### BRIEF DESCRIPTION OF THE DRAWINGS

FIG. 1 shows the effect of the ratio of final detergent concentration (% v/v Triton X-100) to plant cell concentration (R) on the levels of polyclonal antibody reactive (PAb) HBsAg detectable, per gram fresh weight (FW), in crude lysates of stationary phase transgenic tobacco cell lines. ELISA standard deviations for all data points were less than 10% of the mean.

FIGS. 2A-B compare the effect of detergent (% v/v Triton X-100) to plant cell concentration (R) on polyclonal antibody reactive (PAb) HBsAg and monoclonal antibody reactive (MAb) HBsAg levels detectable in crude cell lysates. In FIG. 2A, there is a fold increase in antigen levels relative to buffer lacking detergent for 2 different trials using soybean W82 HB155-37 suspended cells. FIG. 2B shows the fold increase in antigen levels relative to buffer lacking detergent for 2 different trials using tobacco NT1 HB155-18 suspended cells. Standard deviations for all PAb-HBsAg data points were less that 6% of the mean. Due to the cost of the MAb-assay, multiple samples were not analyzed.

FIG. 3 is a comparison of polyclonal reactive (PAb) HBsAg detectable in crude cell lysates of transgenic soybean W82 HB37 cells, following extraction with either a phosphate-buffered saline (PBS)- or carbonate-buffered (C/BC) solution. Extraction was carried out either with no detergent or in the presence of the optimal detergent (% v/v Triton X-100) to plant cell concentration ratio (R 0.6).

FIG. 4 shows the effect of the detergent (% v/v Triton X-100) to plant cell concentration ratio (R) on titers of total small hepatitis B surface protein (p24^{s}) present in crude cell lysates of transgenic soybean cells. Total p24^{s} was quantified by reverse phase-HPLC. Three different cell mass to buffer ratios were tested and sonication was employed to lyze the cells.

FIGS. 5A-B show the effect of sodium ascorbate on levels of monoclonal antibody reactive (MAb) and polyclonal antibody reactive (PAb) HBsAg detectable in crude cell lysates. Other buffer components were held constant and the optimal detergent to plant cell concentration ratio (R=0.6) employed. FIG. 5A shows a fold change in MAb- and PAb-HBsAg detectable for tobacco NT1 HB155-18 cell lysates. Antigen titers at 0% sodium ascorbate: MAb, 0.1 μg/g FW; PAb, 4.8 μg/g FW. FIG. 5B shows a fold change in MAb- and PAb-HBsAg detectable in soybean HB155-37 cell lysates. Antigen titers at 0% sodium ascorbate: MAb, 1.3 μg/g FW; PAb, 42.5 μg/g FW. FIG. 5C shows the effect of sodium ascorbate (NA), with or without a nitrogen environment (N2), on PAb- and MAb-HBsAg titers in crude transgenic soybean W82 HB155-37 cell lysates.

FIGS. 6A-D show the influence of the detergent to plant cell concentration ratio (R) on the stability of polyclonal antibody reactive (PAb) HBsAg in crude cell suspension extracts stored at 4° C. FIG. 6A, transgenic soybean W82 HB155-37 cell crude lysate; initial PAb-HBsAg concentration was 3.05 μg/ml. FIG. 6B shows the transgenic tobacco NT1 HB155-18 crude cell lysate; initial PAb-HBsAg concentration was 0.37 μg/ml. For both profiles, two sodium ascorbate levels (0.5% and 2%) were simultaneously tested and found to give similar results, so all data points were pooled and averaged. FIG. 6C shows stability as a function of R, of HBsAg in soybean cell W82 HB155-37 lysates after storage for 35 days at 4° C. FIG. 6D depicts an analysis of p24^{s} present in fresh and 35 day old transgenic soybean W82 HB155-37 cell lysates, obtained over a range of R values, by Western blot under reducing conditions.

FIGS. 7A-B show the effect of sodium ascorbate level on antigen stability in crude cell lysates of transgenic tobacco cells. FIG. 7A shows the change in polyclonal antibody reactive (PAb) HBsAg relative to initial level (330±20 ng/ml) with storage at 4° C. FIG. 7B shows the change in total soluble protein (TSP) relative to initial level (295±30 μg/ml) with storage at 4° C. Samples were extracted at a detergent to plant cell concentration ratio (R) of 0.6. Error bars, omitted for clarity, never exceeded 110% of mean value. Parallel studies performed with transgenic soybean W82 HB155-37 cell lysates yielded similar results.

FIG. 8 shows the effect of storage (35 days at 4° C.) on the level of polyclonal antibody reactive (PAb) HBsAg in crude cell extracts from μtomato HB120-204 fruits, tomato HB117-25 fruits, and potato HB114-16 tubers. Samples were extracted at a detergent to plant cell concentration ratio (R) of 0.6 with 2% w/v sodium ascorbate present. Sodium ascorbate concentrations over the range 0% to 20% w/v were tested and gave similar results. The initial PAb-HBsAg content for each tissue was: μtomato-HB120-204 fruits, 70±4 μg/g FW; tomato HB117-25 fruits, 9±1 μg/g FW; potato HB114-16 tubers, 88±5 μg/g FW.

FIG. 9A shows the effect of the addition of skim milk (final concentration 5% w/v) on HBsAg stability in crude transgenic soybean W82 HB155-37 cell lysates with storage at 4° C. Control samples were diluted with the equivalent volume of PBS. Final total protein concentrations for skim milk-containing and control solutions were 6.5 mg/ml. Polyclonal (PAb) and monoclonal (MAb) antibody reactive HBsAg profiles for samples extracted at a detergent to plant cell concentration ratio (R)=1.4. Error bars never exceeded ±8% of the mean value. FIG. 9B is a Western blot (reducing conditions) of select samples at various ages, extracted at R=1.4 Symbols; −, control; +, 5% w/v skim milk; C, PBS control; SM, skim milk. FIG. 9C: Tobacco NT1 HB155-18 100 day stability study with 5% w/v skim milk PAb-HBsAg measured.

FIGS. 10A-D show a comparison of stability of tobacco-derived HBsAg in crude cell lysates containing either skim milk or components thereof. Final detergent to cell concentration ratio in samples ranged from 1.3 to 1.6. In FIG. 10A, the samples contain skim milk; final total protein concentrations ranged from 0.2 mg/ml (0% skim milk) to 35 mg/ml (20% skim milk). In FIG. 10B, the samples contain lactose; concentrations up to 35% w/v were tested with identical results. Samples contain whey protein concentrate (WPC); total protein level was 48 mg/ml total protein at 16% WPC. Casein was tested up to 10% w/v (24 mg/ml TSP) and yielded similar results to WPC. Tobacco cells were extracted using a Waring blender, centrifuged to remove cell debris, and the supernatants were combined with the various excipients. FIG. 10C shows the initial Pab-HBsAg and Pab-HBsAg levels after storage for 40 days at 4° C. The crude tobacco NT1 HB155-18 protein extract was combined with PBS (0% control) or casein in PBS at various concentrations. The final detergent to cell concentration ratio (R) in the samples was 1.4 and extracts were prepared using a Waring blender. The initial HBsAg levels in the presence of skim milk was 270+/−26 μg/L. FIG. 10D shows the effect of 5% milk on the initial level of MAb-HBsAg, determined by the Auszyme assay, in a crude protein extract from tobacco NT1 HB155-18 suspension culture. The final detergent to cell concentration ratio (R) in the sample was 1.4 and the extract was prepared using a Waring blender.

FIGS. 11A-B show the increase in monoclonal antibody reactive (MAb) HBsAg in crude cell lysates stored at 4° C. In FIG. 11A, transgenic soybean W82 HB155-37 cells were extracted at a detergent to cell concentration ratio (R)=0.6. Initial PAb-HBsAg level was 47±3 μg/g FW. With 0.5% Na Ascorbate, MAb-HBsAg titers reached 25 μg/g FW by Day 40. At 2% Na Ascorbate, MAb-HBsAg titers reached 19 μg/g FW by Day 40. In FIG. 1B, transgenic tobacco NT1 HB155-18 cells were extracted at an R ratio (R)=0.6. Initial PAb-HBsAg level was 5.6±0.4 μg/g FW. With 0.5% Na Ascorbate, MAb-HBsAg titers reached 5 μg/g FW by Day 31. At 2% Na Ascorbate, MAb-HBsAg titers reached 2.9 μg/g FW by Day 31.

FIGS. 12A-B show the effect of sodium ascorbate concentration on the increase in monoclonal antibody reactive (MAb) HBsAg levels and in vitro disulfide bond formation for transgenic soybean W82 HB155-37 cell extracts stored at 4° C. FIG. 12A shows the MAb-HBsAg profile over an 82 day period for samples lacking sodium ascorbate or containing anti-oxidant at concentrations from 0.5% to 20% w/v. FIG. 12B shows the non-reducing Western blot of initial and 3 day old samples (both containing 0.5% (w/v) sodium ascorbate) and 33 day old samples over the full range of antioxidant concentrations tested. Symbols; M, monomer; D, dimer; T, trimer; Tt, tetramer; O, higher order oligomers.

FIGS. 13A-B show the effect of storage temperature on antigenicity and protein levels in crude transgenic soybean W82 HB155-37 cell extracts (0% Na ascorbate, detergent to plant cell concentration ratio=0.6) after 35 days storage at 4° C. FIG. 13A shows the total soluble protein (TSP), polyclonal (PAb), and monoclonal (MAb) antibody reactive HBsAg in initial extract and after storage. The initial PAb-HBsAg level in the extracted material was 35 μg/g FW. FIG. 13B shows the Western blot under reducing conditions of initial and aged samples. Greater antigen precipitation is evident with increasing storage temperature.

FIGS. 14A-B show the effect of extraction buffer pH together with sodium ascorbate on both the formation of monoclonal antibody reactive (MAb) HBsAg and the extent of in vitro disulfide bonding in crude extracts of transgenic soybean cells W82 HB155-37. FIG. 14A shows the MAb-reactive epitope formation and stability in crude cell lysate stored at 4° C. Legend; C/BC, carbonate/bicarbonate buffer; PBS, phosphate buffered saline; NA, sodium ascorbate (final concentration 2% w/v). The detergent to plant cell concentration ratio (R) was 0.6. FIG. 14B shows the non-reducing Western blot of initial extracts (Day 0) and samples after 1 and 42 days of storage at 4° C. Samples analyzed by Western blot contained no sodium ascorbate. Symbols; M, monomer; D, dimer; O, oligomer.

FIGS. 15A-B show an analysis of soybean HB155-37 crude cell extracts obtained at different detergent concentration to cell concentration (R) ratios. Cell culture material was extracted under 2 detergent conditions, (R=0.68) or with a buffer containing an eight fold lower Triton X-100 concentration (R=0.08) (A). The HBsAg detectable by polyclonal ELISA differed by a factor of two whereas the Western blot band intensity for the extracts differed by only 5% (B).

FIGS. 16A-B show the determination of the presence of virus-like particles (VLPs) in soybean HB155-37 crude cell extracts. Cell samples were extracted under 2 buffer conditions, detergent concentration to cell concentration ratio, R=0.68, or with a buffer containing an eight fold lower TX-100 concentration (R=0.08) and run on a 5-50% sucrose gradient. (1) Comparison of R=0.68 soybean extract profile with that of the yeast standard. (2) Comparison of sucrose gradient profiles obtained under the different detergent conditions. Note the expected VLP peak at R=0.68 and its absence at the lower detergent concentration.

FIGS. 17A-B show the distribution of p24^{s} monomer in sucrose gradients of soybean HB155-37 crude cell lysates, prepared at two different detergent concentration to cell concentration (R) ratios. Western blots were run under non-reducing conditions. The (•) indicates p24^{s} trimers and tetramers.

### DETAILED DESCRIPTION OF THE INVENTION

The present invention relates to a composition, which includes a stabilized hepatitis B surface antigen which can contain a milk protein and/or a milk protein component.

The present invention relates to an oral vaccine for treatment of hepatitis B, which includes a composition formed from a stabilized hepatitis B surface antigen which can contain a milk protein and/or a milk protein component and a carrier.

The present invention relates to a method of immunizing a subject against hepatitis, which comprises administering to the subject a stabilized composition of hepatitis B surface antigen which can contain a milk protein and/or a milk protein component.

The present invention relates to a method of producing a stabilized hepatitis B surface antigen protein, which comprises providing a cell culture suspension containing hepatitis B surface antigen and extracting said hepatitis B surface antigen with a buffer that can contain a milk protein and/or a milk protein component to yield a stabilized hepatitis B surface antigen protein extract.

The present invention relates to a method of administering a composition of a hepatitis B surface antigen which can be stabilized with a milk protein and/or a milk protein component, where the stabilized composition of hepatis B surface antigen is administered orally, these two administration routes apply exclusively to purified HBsAg preparations.

The hepatitis B surface antigen, which is suitable for use in the present invention, is a 226 amino acid protein, with a molecular weight of approximately 25 kDa. It is a membrane bound protein with 4 transmembrane regions. In its native form, the HBsAg protein decorates the surface of 22 nm Virus Like Particles (VLPs). These VLPs are composed of approximately 75% protein, 25% lipid, with extensive disulfide bridging between the monomer subunits to generate oligomers of 14-16 units.

Moreover, several different subtypes of HBsAg exist, which must all possess the group specific a determinant. Any vaccine against the hepatitis B virus must display the correctly folded a determinant as antibodies against this epitope are required to generate an effective immune response.

The hepatitis B surface antigen suitable for use in the present invention has a nucleotide sequence and an amino acid sequence as described in Okamoto et al., “Typing Hepatitis B Virus by Homology in Nucleotide Sequence: Comparison of Surface Antigen Subtypes,” J. Gen. Virol. 69:2575 (1988), which is hereby incorporated by reference in its entirety. The following references also provide a historical background toward efforts in understanding the hepatitis antigen: Vyas and Shulman, Science 170:332 (1970); Rao and Vyas, Nature New Biology 241:240 (1973); Rao and Vyas, Microbios. 9:239 (1974); Rao and Vyas, Microbios. 10:233 (1974); Peterson et al., Proc. Nat'l Acad. Sci. USA 74:1530 (1977); Vyas et al. ed., Viral Hepatitis Proceedings of UCSF Symposium, Franklin Institute Press, Philadelphia (1978); Valenzuela et al., Nature 280:815 (1979); Chamay et al., Nucleic Acid Res. 7:355 (1979), which are hereby incorporated by reference in its entirety).

Further, the amino acid sequence of the “a” determinant of hepatitis B surface antigen and peptides with specific amino acid sequences and variants thereof, similar to that group specific “a” determinant is described in U.S. Pat. No. 5,531,990 to Thanavala, et al. which is hereby incorporated by reference in its entirety. See also Carman, et al., “Molecular Variants of Hepatitis B Virus,” Viral Hepatitis 141-172 (1998), which is hereby incorporated by reference in its entirety.

The hepatitis B surface antigen (HBsAg), which was used as a model system of the present invention, was expressed in plant cell tissue culture. This complex antigen consists of membrane-associated small surface antigen proteins (p24^{s}) disulfide cross-linked to yield dimers and higher multimers. Although the total p24^{s} extracted from plant cells was relatively unaffected by detergent concentration, the quantification of antigenically reactive product depended strongly on the ratio of detergent to cell concentration.

In accordance with the present invention, the hepatitis B surface antigen suitable for use in the present invention may be produced by conventional plant genetic engineering techniques. By techniques as described herein below, a model hepatitis B surface antigen (HBsAg) system used in the present invention may be expressed or produced in plant cell suspensions, cell tissue culture, etc. and in different HBsAg physical forms. Different HBsAg physical forms, which are suitable for use in the present invention, may include, but are not limited to antigen protein which is or may be in the form of the serum-derived antigen.

In general, methods of making recombinant plant cell(s) involve the introduction of recombinant molecules (e.g., heterologous or not normally present foreign DNA construct, such as a hepatitis B surface antigen) into host cells (e.g., host cells of plant(s), plant tissues, etc.) via specific types of transformation. Thus, a DNA construct contains necessary elements for the transcription and translation in plant cells of an heterologous DNA molecule.

The DNA construct of the present invention also includes an operable 3′ regulatory region, selected from among those which are capable of providing correct transcription termination and polyadenylation of mRNA for expression in plant cells, operably linked to the a DNA molecule which encodes for a protein of choice. A number of 3′ regulatory regions are known to be operable in plants. Exemplary 3′ regulatory regions include, without limitation, the nopaline synthase 3′ regulatory region (Fraley, et al., “Expression of Bacterial Genes in Plant Cells,” Proc. Nat'l Acad. Sci. USA, 80:4803-4807 (1983), which is hereby incorporated by reference in its entirety) and the cauliflower mosaic virus 3′ regulatory region (Odell, et al., “Identification of DNA Sequences Required for Activity of the Cauliflower Mosaic Virus 35S Promoter,” Nature, 313(6005):810-812 (1985), which is hereby incorporated by reference in its entirety). Virtually any 3′ regulatory region known to be operable in plants would suffice for proper expression of the coding sequence of the DNA construct of the present invention.

The DNA molecule, the promoter, and a 3′ regulatory region can be ligated together using well known molecular cloning techniques as described in Sambrook et al., Molecular Cloning: A Laboratory Manual, Second Edition, Cold Spring Harbor Press, NY (1989), which is hereby incorporated by reference in its entirety.

The DNA construct can also include a DNA molecule encoding a secretion signal. A number of suitable secretion signals are known in the art and others are continually being identified. The secretion signal can be a DNA leader which directs secretion of the subsequently translated protein or polypeptide, or the secretion signal can be an amino terminal peptide sequence that is recognized by a host plant secretory pathway. The secretion-signal encoding DNA molecule can be ligated between the promoter and the protein-encoding DNA molecule, using known molecular cloning techniques as described in Sambrook et al., Molecular Cloning: A Laboratory Manual, Second Edition, Cold Spring Harbor Press, NY (1989), which is hereby incorporated by reference in its entirety.

A further aspect of the present invention relates to an expression system that includes a suitable vector containing a DNA construct. In preparing the DNA construct for expression, the various DNA sequences may normally be inserted or substituted into a bacterial plasmid. Any convenient plasmid may be employed, which will be characterized by having a bacterial replication system, a marker which allows for selection in a bacterium and generally one or more unique, conveniently located restriction sites. Numerous plasmids, referred to as transformation vectors, are available for plant transformation. The selection of a vector will depend on the preferred transformation technique and target species for transformation.

A variety of vectors are available for stable transformation using Agrobacterium tumefaciens, a soilborne bacterium that causes crown gall. Crown gall are characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTI, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes and replacing them with a gene of interest, it is possible to transfer foreign DNA into the plant without the formation of tumors or the multiplication of Agrobacterium tumefaciens (Fraley, et al., “Expression of Bacterial Genes in Plant Cells,” Proc. Nat'l Acad. Sci., 80:4803-4807 (1983), which is hereby incorporated by reference in its entirety).

Further improvement of this technique led to the development of the binary vector system (Bevan, M., “Binary Agrobacterium Vectors for Plant Transformation,” Nucleic Acids Res. 12:8711-8721 (1984), which is hereby incorporated by reference in its entirety). In this system, all the T-DNA sequences (including the borders) are removed from the pTi, and a second vector containing T-DNA is introduced into Agrobacterium tumefaciens. This second vector has the advantage of being replicable in E. coli as well as A. tumefaciens and contains a multiclonal site that facilitates the cloning of a transgene. An example of a commonly used vector is pBin 19 (Frisch et al., “Complete Sequence of the Binary Vector Bin19,” Plant Molec. Biol. 27:405-409 (1995), which is hereby incorporated by reference in its entirety). Any appropriate vector now known or later described for plant transformation is suitable for use with the present invention.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures, including procaryotic organisms and eukaryotic cells grown in tissue culture.

A further aspect of the present invention includes a host cell which includes a DNA construct of the present invention. As described more fully hereinafter, the recombinant host cell can be either a bacterial cell (e.g., Agrobacterium) or a plant cell. In the case of recombinant plant cells, it is preferable that the DNA construct is stably inserted into the genome of the recombinant plant cell.

The DNA construct can be incorporated into cells using conventional recombinant DNA technology. Generally, this involves inserting the DNA construct into an expression vector or system to which it is heterologous (i.e., not normally present). As described above, the DNA construct contains the necessary elements for the transcription and translation in plant cells of the heterologous DNA molecule.

Once the DNA construct of the present invention has been prepared, it is ready to be incorporated into a host cell. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable host cells include, but are not limited to, bacteria, virus, yeast, mammalian cells, insect, plant, and the like. Preferably the host cells are either a bacterial cell or a plant cell.

Accordingly, another aspect of the present invention relates to a method of making a recombinant plant cell and/or plant cell cultures, tissues, suspensions, etc. Basically, this method is carried out by transforming such a plant cell and/or plant cell cultures, tissues, suspensions, etc. with a DNA construct of the present invention under conditions effective to yield transcription of the DNA molecule. Preferably, the DNA construct of the present invention is stably inserted into the genome of the recombinant plant cell as a result of the transformation.

One approach to transforming plant cells and/or plant cell cultures, tissues, suspensions, etc. with a DNA construct of the present invention is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford, et al., which are hereby incorporated by reference in its entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells and/or plant cell cultures, tissues, suspensions, etc. Other variations of particle bombardment, now known or hereafter developed, can also be used.

Another method of introducing the gene construct of the present invention into a host cell is fusion of protoplasts with other entities, either minicells, cells, lysosomes, or other fusible lipid-surfaced bodies that contain the chimeric gene (Fraley, et al., Proc. Natl. Acad. Sci. USA, 79:1859-63 (1982), which is hereby incorporated by reference in its entirety).

The DNA construct of the present invention may also be introduced into the plant cells and/or plant cell cultures, tissues, suspensions, etc. by electroporation (Fromm, et al., Proc. Natl. Acad. Sci. USA, 82:5824 (1985), which is hereby incorporated by reference in its entirety). In this technique, plant protoplasts are electroporated in the presence of plasmids containing the DNA construct.

Another method of introducing the DNA construct into plant cells and/or plant cell cultures, tissues, suspensions, etc. is to infect a plant cell with Agrobacterium tumefaciens or Agrobacterium rhizogenes previously transformed with the DNA construct. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the Gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

Heterologous genetic sequences such as a DNA construct of the present invention can be introduced into appropriate plant cells by means of the Ti plasmid of A. tumefaciens or the Ri plasmid of A. rhizogenes. The Ti or Ri plasmid is transmitted to plant cells on infection by Agrobacterium and is stably integrated into the plant genome (Schell, J., Science, 237:1176-83 (1987), which is hereby incorporated by reference in its entirety).

Plant tissue and/or plant cell cultures, tissues, suspensions, etc. suitable for transformation also include, but are not limited to leaf tissue, root tissue, menstems, zygotic and somatic embryos, megaspores and anthers.

After transformation, the transformed plant cells and/or plant cell cultures, tissues, suspensions, etc. can be selected and regenerated.

Preferably, transformed cells are first identified using a selection marker simultaneously introduced into the host cells along with the DNA construct of the present invention. Suitable selection markers include, without limitation, markers coding for antibiotic resistance, such as the nptII gene which confers kanamycin resistance (Fraley, et al., Proc. Natl. Acad. Sci. USA, 80:4803-4807 (1983), which is hereby incorporated by reference in its entirety) and the dhfr gene, which confers resistance to methotrexate (Bourouis et al., EMBO J. 2:1099-1104 (1983), which is hereby incorporated by reference in its entirety). A number of antibiotic-resistance markers are known in the art and others are continually being identified. Any known antibiotic-resistance marker can be used to transform and select transformed host cells in accordance with the present invention. Cells or tissues are grown on a selection media containing an antibiotic, whereby generally only those transformants expressing the antibiotic resistance marker continue to grow. Similarly, enzymes providing for production of a compound identifiable by color change are useful as selection markers, such as GUS (β-glucuronidase), or luminescence, such as luciferase.

Also suitable as selection markers for the present invention are genes that cause the overproduction of a plant product, which may be in the form of plant cell culture, tissues, suspensions, etc. such as the cytokinin-synthesizing ipt gene from A. tumefaciens. Localized over-production of cytokinin can be determined by known methods, such as ELISA assay (Hewelt et al., “Promoter Tagging with a Promoterless ipt Gene Leads to Cytokine-induced Phenotypic Variability in Transgenic Tobacco Plants: Implications of Gene Dosage Effects,” Plant J. 6:879-91 (1994), which is hereby incorporated by reference in its entirety). The selection marker employed will depend on the target species; for certain target species, different antibiotics, herbicide, or biosynthesis selection markers are preferred.

Once a recombinant plant cell and/or plant cell cultures, tissues, suspensions, etc. has been obtained, it is possible to regenerate a full-grown plant therefrom.

The transgenic plant and/or plant cell cultures, tissues, suspensions, etc. includes a DNA construct of the present invention, wherein the DNA promoter induces transcription of the protein-encoding DNA molecule in response to developmental activation of the promoter. Preferably, the desired heterologous DNA construct is stably inserted into the genome of the transgenic plant of the present invention.

Plant regeneration from cultured protoplasts is described in Evans et al., Handbook of Plant Cell Cultures, Vol. 1: (MacMillan Publishing Co., New York, 1983); and Vasil I. R. (ed.), Cell Culture and Somatic Cell Genetics of Plants, Acad. Press, Orlando, Vol. 1, 1984, and Vol. III (1986), which are hereby incorporated by reference in their entirety.

Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the DNA construct is stably incorporated in transgenic plants, and/or plant cell cultures, tissues, suspensions, etc., it can be transferred to other plants by sexual crossing or by preparing cultivars. With respect to sexual crossing, any of a number of standard breeding techniques can be used depending upon the species to be crossed. Cultivars can be propagated in accord with common agricultural procedures known to those in the field.

Once transgenic plants of this type are produced, the plants and/or plant cell cultures, tissues, suspensions, etc. themselves can be cultivated in accordance with conventional procedures. Alternatively, transgenic seeds are recovered from the transgenic plants. The seeds can then be planted in the soil and cultivated using conventional procedures to produce transgenic plants.

Techniques for recovery of the product of expression of any heterologous DNA of choice, which may be derived from plants and or plant cell culture, tissues, suspensions, etc., are known to those skilled in the art.

Some general definitions pertinent to the present invention, such as different plant types, cell cultures, suspensions, tissues, etc. and corresponding or related definitions of materials suitable for use in the present invention follow.

In accordance with the present invention, a transgenic plant is a plant that 10 contains and expresses DNA encoding an HBsAg antigen protein, that was not pre-existing in the plant prior to the introduction of the DNA into the plant.

Suitable transgenic plant material is any plant matter, including, but not limited to cells, cell cultures, all suspensions, protoplasts, tissues, leaves, stems, fruit and tubers both natural and processed, containing and expressing DNA encoding an HBsAg antigen protein, that was not pre-existing in the plant or corresponding cells, cell suspensions, tissues, etc. prior to the introduction of the DNA into the plant.

Further, plant material includes processed derivatives thereof including, but not limited to, food products, food stuffs, food supplements, extracts, concentrates, pills, lozenges, chewable compositions, powders, formulas, syrups, candies, wafers, capsules, and tablets.

In accordance with the present invention, a plant tissue is any tissue of a plant in its native state or in cell culture, suspension, etc. This term includes, without limitation, whole plants, plant cells, plant organs, plant seeds, protoplasts, callus, cell cultures, and any group of plant cells organized into structural and/or functional units. The use of this term in conjunction with, or in the absence of, any specific type to plant tissue as listed above or otherwise embraced by this definition is not intended to be exclusive of any other type of plant tissue.

In accordance with the present invention, an edible plant material includes a plant or any material obtained from a plant which is suitable for ingestion by mammal or other animals including humans. This term is intended to include raw plant material that may be fed directly to animals or any processed plant material that is fed to animals, including humans. Materials obtained from a plant are intended to include any component of a plant which is eventually ingested by a human or other animal.

In particular, plants (as well as corresponding plant cell suspensions, plant tissues, plant extracts, etc.) suitable for use in the present invention, such as for use as host cells in the expression of hepatitis B surface antigen, include, but are not limited to tobacco, Arabidopsis thaliana, soybean, mustard plant, tomato, alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, sorghum, sugarcane, and banana.

Suitable plant extracts, may include, but are not limited to those derived from tobacco NT1 (Nicotiana tobacum) cell suspension cultures (line NT 1 HB155-18), soy bean (Glycine max) (line W82 HB155-37), potato tuber (Solanum tuberosum variety “Frito Lay 1607”) (line HB114-16), tomato fruit (Lycopersicon esculentum cv monor) (line HB 117-25), tomato fruit (Licopersicon esculentum cv MicroTom) (line HB120-204), and carrot (Daucos carota) cell suspension cultures.

The present invention further relates to a method of producing a stabilized hepatitis B surface antigen protein, which includes providing a plant cell suspension with expressed hepatitis B surface antigen. This is carried out by maintaining the hepatitis B surface antigen containing cell culture suspension at a temperature range of 20° C. to 32° C. for growth conditions and the plant extract at a temperature range of −20° C. to 20° C., but preferably between 2° C. and 10° C., for storage conditions.

In accordance with the present invention, methods for producing stabilized hepatitis B surface antigens of the present invention also includes contacting such hepatitis B surface antigen plant cell suspensions, plant extracts, etc. with stabilizing agents, such as milk samples (which contain a protein or a corresponding component) or extraction buffers under conditions that stabilize such suspensions or extracts containing the expressed antigen protein.

Purification of crude hepatitis B surface antigen plant extracts (i.e., as prepared by the methods described herein) is not necessary as the use of milk protein and components thereof aid in the stabilization of the expressed HBsAg protein. In such methods, the expressed hepatitis B surface antigen protein plant cell suspension(s), plant extract(s), plant tissue(s), etc. are stabilized with a milk protein and/or a milk protein component, which may include, but is not limited to lactose, casein, sodium caseinate, whey protein and minerals or an extraction buffer (i.e., which may include the aforementioned components) to yield a stabilized hepatitis B surface antigen protein composition. In the method of the present invention, non-hepatitis B surface antigen components, present in the crude plant extracts, may be separated from the stabilized hepatitis B surface antigen protein by art known separation or extraction techniques.

In addition, the following materials and/or factors are important in the stability and in vitro assembly of the hepatitis B Surface Antigen compositions produced by methods of the present invention.

Milk proteins or isolated components thereof (i.e., in solutions, dried forms, etc.), which may be also be components of extraction buffers, are important materials for the stabilization of hepatitis B surface antigen proteins of the present invention.

Milk proteins suitable for use in the present invention are conventionally known in the art to include a collodial dispersion of casein micelles and soluble whey proteins. Casein micelles are extremely sensitive to changes in ionic environment and readily aggregate with increased concentration of calcium and magnesium ion. Due to the long history of their inclusion in the human diet and the relative ease with which they can be isolated, naturally occurring milk components have been studied for many years (Swaisgood, H. E., Developments in Dairy Chemistry—I: Chemistry of Milk Protein, Applied Science Publishers, NY (1982), which is hereby incorporated by reference in its entirety).

Types of milk proteins suitable for use in the present invention which stabilize the hepatitis B surface antigen, may include and are not limited to soy milk proteins or skim milk proteins. A particularly preferred type of milk used in the present invention is skim milk. Skim milk is a complex mixture of proteins, lactose and minerals, which may include, but is not limited to a group consisting of lactose, casein or sodium caseinate, whey protein, and minerals.

The approximate composition of skim milk is shown in Table 1.

TABLE 1
Skim Milk Components

| Component | % by weight |
|---|---|
| Lactose | 56 |
| Casein | 29 |
| Whey protein (WPC) | 7 |
| Minerals | 8 |

Suitable for use in the present invention are any conventional art known techniques for the isolation of and/or production of milk protein components and/or corresponding forms thereof.

For example, chemical and physical properties of milk and/or corresponding components thereof, uses and isolation of milk components and techniques thereof are described in detail in U.S. Pat. No. 5,756,687 to Denman et al., which is hereby incorporated by reference in its entirety.

Additional traditional methods of isolating a protein of interest from milk include initial fractionation of the major milk components by either sedimentation (Swaisgood, H. E., Developments in Dairy Chemistry—I: Chemistry of Milk Protein, Applied Science Publishers, NY (1982), which is hereby incorporated by reference by its entirety), precipitation (U.S. Pat. No. 4,644,056 to Kothe et al.; Groves, M. L. et al., Biochem. et. Biophys. Acta. 844:105-112 (1985); and McKenzie, H. A., Milk Proteins: Chemistry and Molecular Biology, Academic Press, NY, p. 88 (1971), which are hereby incorporated by reference in their entirety), or enzymatic coagulation using rennin or chymotrypsin (Swaisgood, H. E., Developments in Dairy Chemistry—I: Chemistry of Milk Protein, Applied Science Publishers, NY (1982), which is hereby incorporated by reference in its entirety).

In accordance with the present invention, the concentration of the stabilizing agent, i.e., a milk protein or component thereof is selected such that it preserves the functional properties of and stabilizes hepatitis B surface antigen. The milk protein and/or the milk protein component, may be in a powdered form or liquid form.

In accordance with the present invention, routine experimental conditions under which the hepatitis B surface antigen is brought into contact with a stabilizing agents or solutions (i.e., such as milk proteins and/or corresponding components thereof, extraction buffers) to form a stabilized material, include conditions, such as pH, component content of extraction buffers, antioxidants (e.g., sodium ascorbate), detergent (e.g., Triton X-100 or Tween 20) that could affect the stabilization of the hepatitis B surface antigen.

For example, the pH of the milk samples used in hepatitis B surface antigen plant suspensions generally should be adjusted to a level where HBsAg protein is stabilized and retains its solubility and functional properties. In the present invention, the extraction buffer is maintained at a specific pH range of 6.0 to 8.0, preferably 7.4.

An extraction buffer or buffer solution suitable for use in the present invention, includes, but is not limited to, milk protein and/or milk protein component(s). Such milk protein and/or the milk protein component as used to stabilize hepatitis B surface antigen may be present in the buffer solution at the level of 0.5% to 15% by weight volume (w/v), preferably 5% weight per volume (w/v).

An extraction buffer or buffer solution suitable for use in the present invention may further include an antioxidant, which may also contribute to hepatitis B surface antigen extract stability by decreasing polyphenol formation and protein oxidation. Antioxidants suitable for use may include sodium ascorbate and sodium metabisulfite. The antioxidant of the extraction buffer may be present in the buffer solution at a level of 0% to 20% by weight per volume (w/v), most preferably present in the buffer solution at a level of 1.5% to 2% by weight per volume (w/v).

An extraction buffer in accordance with the present invention can utilize any of the detergents identified. Such detergents are preferably utilized at a ratio of final detergent concentration (% v/v) to plant material concentration (grams of fresh weight per ml) of 0.4 to 0.6.

The present invention also relates to methods of stabilization in crude extract preparations, derived from an edible expression source, preferably plants or plant cell suspensions. Such antigen stabilizing processing methods, include use of stabilization agents to form stabilized antigen containing extracts. The stabilized antigen extracts are incorporated into different oral delivery systems or technologies.

The present invention provides for use of conventional art known oral delivery systems. Suitable forms of oral delivery systems for use with the stabilized hepatitis antigen extracts of the present invention and/or corresponding pharmacological basis or associated mechanisms are discussed herein. Currently, oral delivery of plant-derived subunit vaccines has generated considerable interest and a large number of antigens, targeting both enteric and non-enteric diseases, have been successfully expressed (Richter et al., “Transgenic Plants as Edible Vaccines,” Curr Topics Micro Immunol 240:159-176 (1999); Streatfield et al., “Plant-Based Vaccines-Unique Advantages,” Vaccine 19:2742-2748 (2001), which are hereby incorporated by reference in their entirety). For oral vaccination strategies to be effective, the antigens must reach the gut-associated lymphoid tissue (GALT) intact and in an immunogenic form (Fasano A., “Innovative Strategies for the Oral Delivery of Drugs and Peptides,” Trends Biotechnol 16:152-157 (1998), which is hereby incorporated by reference in its entirety).

A significant concern is the potential for oral tolerance; a systemic, antigen-specific, cellular and humoral immune unresponsiveness (Czerkinsky et al., “Mucosal Immunity and Tolerance: Relevance to Vaccine Development,” Immunol Rev 170:197-222 (1999); Faria et al., “Oral Tolerance: Mechanisms and Therapeutic Applications,” Adv Immunol 73:153-264 (1999); Mowat et al., “ISCOMS—A Novel Strategy for Mucosal Immunization,” Immunol Today 12:383-385 (1987), which are hereby incorporated by reference in their entirety).

The present invention also relates to the protection of protein antigens, which may be protected by encapsulating them in biocompatible and biodegradable microspheres (Eldridge et al., “Biodegradable Microspheres as a Vaccine Delivery System,” Mol Immunol 28:287-294 (1991), which is hereby incorporated by reference in their entirety). Another conventional approach to protecting antigens from gastric digestion is encapsulation by a water-soluble enteric polymethacrylic acid polymer (Ghebre-Sellassie et al., “Eudragit Aqueous Dispersions as Pharmaceutical Controlled Release Devices,” In: McGinity J W, editor. Drugs and the Pharmaceutical Sciences. New York: Marcel Dekker. Vol 79. p 77-97 (1997), which is hereby incorporated by reference in its entirety), which also suitable for use with the present invention. These polymers are stable at low pH (within the stomach) and can be designed to dissolve rapidly over a range of higher pHs (5 to 7), permitting all regions of the small and large intestine to be targeted. Formulation in the presence of sugar is another option suitable for use in the present invention and has been shown to protect a variety of proteins (Crowe et al., “Stabilization of Dry Phospholipid Bilayers and Proteins by Sugars,” Biophys J 242:1-10 (1987), which is hereby incorporated by reference in their entirety). It is also possible to incorporate stabilized HBsAg extracts of the present invention into liposomes prior to formulation (e.g. freeze drying) in the presence of sugar.

Suitable adjuvants may include, but are not limited to, lipid A and derivatives, muramyl peptides, saponins, NBP, DDA, cytokines (such as inerleukins (1, 2, 3, 6, 12), interferon-γ, tumor necrosis factor), and cholera toxin, B subunit.

Suitable carriers for use in the present invention, may include, but are not limited to, delivery systems, such as emulsions, liposomes, ISCOMS, and microspheres.

Suitable vaccine formulations for use in the present invention, may include, but are not limited to, buffer components, salts, preservatives, and stabilizers, antioxidants, or sugars. These additives should not adversely affect vaccine components upon addition, storage, and application.

Suitable preservatives for use in vaccine formulations in the present invention include, but are not limited to, thimerosal, phenoxyethanol, phenol, antibiotics, EDTA, sulfites, and BHA.

Suitable stabilizers for use in the present invention may include, but are not limited to, proteins or other (bio)polymers, carbohydrates, sugar alcohols, formaldehyde (i.e., used as an inactivating agent of toxins and poliovirus and often present in final products where it serves a stabilizer of vaccine components) or any other conventionally known substances that can serve to prolong the vaccine shelf-life and/or minimize deleterious effects of freeze-drying.

An alternative approach for vaccine stabilization suitable for use in the present invention is encapsulation of vaccine components in biodegradable microspheres. This may prevent their degradation by low pH and lytic enzymes in the gastrointestinal tract upon oral administration. It is believed that a minimally processed product, e.g. tissue homogenate or juice extract, which could be further concentrated and formulated into a tablet or gelatin capsule would overcome these concerns. Such a vaccine preparation is acceptable for an orally delivered product as the antigen is derived from an edible source. Therefore, host protein and nucleic acids are non-infectious and commonly encountered by the digestive tract. This approach is not strictly limited to plant-derived vaccines. Currently HBsAg is manufactured in a variety of yeasts, including Saccharomyces cerevisiae (McAleer et al., “Human Hepatitis B Vaccine From Recombinant Yeast,” Nature 307:178-180 (1984); Petre et al., “Development of a Hepatitis B Vaccine From Transformed Yeast Cells,” Postgrad Med J 63:73-81 (1987), which are hereby incorporated by reference in its entirety), and Pichia pastoris (Cregg et al., “High-Level Expression and Efficient Assembly of Hepatitis B Surface Antigen in the Methylotrophic Yeast, Pichia pastoris,” Bio/Technology 5:479-485 (1987); Hardy, et al., “Large-Scale Production of Recombinant Hepatitis B Surface Antigen from Pichia pastoris,” J Biotechnol 77:157-167 (2000), which is hereby incorporated by reference in its entirety). Both organisms are generally regarded as safe (GRAS) (the latter being used for single cell protein production), and as such could provide an “edible” vaccine source, provided the HBsAg present is in an immunogenic form.

An oral vaccine suitable for use in the present invention may be in a form, which may include, but is not limited to, capsule(s), tablet(s), microsphere(s), encapsulated microsphere(s), suspension(s), and lipid-based emulsions.

The present invention also provides for the use of transgenic plants usable as oral vaccines or oral vaccine adjuvants, wherein the plants comprise or express a DNA sequence encoding a Hepatitis B Surface antigen containing protein and/or sequences encoding proteins, protein components thereof and sequences encoding cellular signal and retention polypeptides or proteins, which may include fusions of other antigenic agents.

Moreover, the success of immunization or administration of vaccines of the present invention is not only dependent on the nature of the immunogenic components, but also on their presentation form, such as effective and acceptable adjuvants and delivery systems. Thus, the present invention relates to an oral vaccine for treatment of hepatitis B, which may also include a composition of the present invention and a carrier, such as a pharmaceutically acceptable adjuvants, carriers, or excipients.

The present invention also relates to alternate methods of administering vaccines of the invention. Whichever mode of introduction of the vaccine to the mammalian subject is selected, it will be understood by those skilled in the art of vaccination that the selected mode must achieve vaccination at the lowest dose possible in a dose-dependent manner and, by so doing, elicit serum and/or secretory antibodies against the immunogen of the vaccine with minimal induction of systemic tolerance.

In certain general embodiments, such methods comprise administering a therapeutic amount of the vaccine to a subject, such as a mammal. Exact therapeutic amounts remain to be determined but are estimated to be in the range of 500 μg to 2 mg total HBsAg protein per dose.

The method of administering a composition according to the present invention includes a stabilized composition of hepatitis B surface antigen which is preferably delivered via oral ingestion routes into a subject. Oral administration of the present invention may be achieved by oral vaccine(s), controlled release preparation(s), and sublingual administration.

The present invention also relates to a method of increasing immunogenicity of a hepatitis B surface antigen. This involves providing a cell culture medium comprising a hepatitis B surface antigen and extracting the hepatitis B surface antigen with a buffer containing a pH of 7 to 12 to yield an extract. The extract is stored at 0 to 10° C., preferably for up to 40 days, so that the hepatitis B surface antigen has an increased immunogenicity. This procedure permits the fraction of antigen displaying determinants necessary for immunogenicity to increase. In carrying out this aspect of the invention, a detergent containing buffer can be utilized.

### EXAMPLES

The Examples set forth below are for illustrative purposes only and are not intended to limit, in any way, the scope of the present invention.

### Example 1

### Sources of Experimental Materials Used in Present Invention

Hepatitis B surface antigen: HBsAg is a 226 amino acid protein, with a molecular weight of approximately 25 kDa. It is a membrane bound protein with 4 transmembrane regions. In its native form the HBsAg protein decorates the surface of 22 nm Virus Like Particles (VLPs). These VLPs are composed of approximately 75% protein, 25% lipid, with extensive disulfide bridging between the monomer subunits to generate oligomers of 14-16 units. Several different subtypes of HBsAg exist; however, they all possess the group specific a determinant. Any vaccine against the Hepatitis B virus must display the correctly folded a determinant as antibodies against this epitope are required to generate an effective immune response.

Cell lines: The following cell lines were used in experiments of the present invention: Tobacco NT1 (Nicotiana tabacum L.) cell-suspension cultures were maintained in medium containing Murashige-Skoog (MS) (Murashige et al., “A Revised Medium for Rapid Growth and Bioassays with Tobacco Tissue Cultures,” Physiol Plant 15:473-497 (1962), which is hereby incorporated by reference) basal salts, 30 g/l sucrose, 0.5 g/L 2-[N-morpholino]ethanesulfonic acid (MES), 1 mg/L thiamine-HCl, 100 mg/L inositol, 180 mg/L potassium phosphate and 0.22 mg/L 2,4-D. The cells were subcultured every 7 days by diluting 2 mL packed cell volume (PCV) of the old culture into 50 mL of fresh medium, in a 500 mL Erlenmeyer flask. Soybean (Glycine max L. Merr. cv Williams 82) cell suspension cultures were maintained in medium containing MS basal salts and vitamins, supplemented with 30 g/L sucrose and 0.4 mg/L 2,4-D, and subcultured every 10 days by diluting 2 mL PCV of the old culture in 50 mL fresh medium, in a 250 mL Erlenmeyer flask. Both cell lines were maintained in the dark on an orbital shaker-incubator at 120 rpm and 27° C. Stationary phase cultures of tobacco NT1 line HB155-18 (10-13 days post-subculture) and soybean W82 line HB155-37 (14-20 days post-subculture) were employed. The use of two different cell lines permits generalization of the results obtained for the in vitro production systems.

For use in the present invention, such cells were maintained in an orbital shaker-incubator at 120 rpm and 27° C. The medium used for tobacco NT1 consists of Murashige-Skoog (MS) basal salts supplemented with 30 g/l sucrose, 0.5 g/L 2-[N-morpholino] ethanesulfonic acid, 1 mg/L thiamine-HCl, 100 mg/L inositol, 180 mg/L potassium phosphate and 0.22 mg/L 2,4-D. The cells were subcultured every 7 days by diluting 1.5 ml of the old culture into 50 ml of fresh medium, in a 500 ml Erlenmeyer flask.

For use in the present invention, potatoes (Solanum tuberosum L. cv “Frito Lay 1607”) of transgenic line HB114-16 (Richter, et al., “Production of Hepatitis B Surface Antigen in Transgenic Plants for Oral Immunization,” Nat. Biotechnol 18:1167-1171 (2000), which is hereby incorporated by reference in its entirety) had been in cold storage (4° C.) for four months prior to analysis.

For use in the present invention, for the tomato fruit, Lycopersicon esculentum Mill. cv Momor, transgenic line HB117-25 and cv Micro-Tom, transgenic line HB120-204 were processed within 5 days after harvesting and were red ripe. Construct HB117 employed the CaMV double 35S promoter, and construct HB120 employed the fruit ripening specific E8 promoter. Potato tuber and tomato fruit were tested to determine how candidate whole plant systems compared to the in vitro cultured plant material.

Example of Transformation: Agrobacterium tumefaciens mediated transformation was used to introduce the native HBsAg gene into the tobacco cell culture. Biolistic transformation was used to introduce the native HBsAg gene into the soybean cell line.

Example of Vectors Used For Stable Transformation: For use in the present invention the HB155 expression vector was similar to HB104 (Richter, et al., “Production of Hepatitis B Surface Antigen in Transgenic Plants for Oral Immunization,” Nat. Biotechnol 18:1167-1171 (2000), which is hereby incorporated by reference in its entirety), except that the Gelvin promoter (Ni, et al., “Strength and Tissue Specificity of Chimeric Promoters Derived From the Octopine and Mannopine Synthase Genes,” Plant J. 7:661-676 (1995), which is hereby incorporated by reference in its entirety) replaced the CaMV (cauliflower mosaic virus) double 35S promoter and TEV (tobacco etch virus) 5′ untranslated leader.

### Example 2

### General Experimental Procedures and Methods Used in Present Invention: Sample Preparation, and Extraction Conditions

Plant Material Sampling: For suspension cultures, cells were vacuum filtered through MiraCloth (Calbiochem, La Jolla, Calif.), rinsed once with distilled water, and re-filtered. Weighed samples were transferred to 2 ml screw-cap microcentrifuge tubes and stored at −70° C. until extraction. Potato tubers were peeled and a segment cut, diced finely with a single-edge razor blade, weighed, and samples stored at −70° C.

Extraction procedure: For the enzyme-linked immunosorbent assays (ELISA's) and Western blot analysis, extraction of suspended cells and potato was performed on frozen samples (typically 70 mg) by homogenization for 30 seconds using a Fastprep FP120 (Bio101, Vista, Calif.), in a coldroom. To each tube 1 ml of cold extraction buffer (1× Dulbecco's phosphate buffered saline (PBS), pH 7.4 (Pierce, Rockford, Ill.), 10 mM ethylenediaminetetraacetic acid, Triton X-100, sodium ascorbate) was added, and lysis was performed using a single 1/4″ ceramic bead (a 5/16″ ceramic cylinder was also included for potato tuber). Triton X-100 and sodium ascorbate concentrations were varied.

Tomato fruit extraction was performed immediately following sampling; flesh from the outer wall of the pericarp was weighed (350 mg) and extracted in 5.35 ml of extraction buffer using a Tenbroeck borosilicate glass homogenizer. For reverse phase high performance liquid chromatography (RP-HPLC), 400 mg tissue samples were extracted with 1 to 3 volumes buffer using a Microtip Sonic Disruptor (Tekmar, Cincinnati, Ohio) (3×10 second cycles, 40% amplitude). All sample lysates were centrifuged for 3 minutes at 10,000 rpm and stored on ice prior to analysis

### Example 3

### Measurement of Total p24^{s} Monomer Levels

Both reverse phase high performance liquid chromatography (RP-HPLC) and Western blot were used to measure total p24^{s} protein in crude cell extract, independent of antigen conformation and extent of disulfide bonding. For RP-HPLC, a HYTACH C18 column (Glycotech, New Haven, Conn.) was employed, following the procedure of O'Keefe and Paiva (O'Keefe, et al., “Assay for Recombinant Hepatitis B Surface Antigen Using Reversed-Phase High-Performance Liquid Chromatography,” Anal. Biochem. 230:48-54 (1995), which is hereby incorporated by reference in its entirety) with the following modifications.

Samples were heated to 95° C. in glass vials (Kimble, Vineland, N.J.) or polyethylene tubes (Robbins Scientific, Sunnyvale, N.J.) with pretreatment buffer (0.1 M Tris pH 8.0, 4% sodium dodecyl sulfate (SDS)/1.3 M dithiothreitol (DTT)/55% v/v 2-β-mercaptoethanol). Polypropylene microcentrifuge tubes were avoided as plasticizers and were leached by the β-mercaptoethanol, which eluted with approximately the same retention time as p24^{s}. 20 μl samples were injected onto the following linear gradient; 0-5 min, 45% solvent B (0.1% trifluoroacetic acid in isoproponal:acetonitrile (80:20)), 5-8 min, 45-95% B; 8-9 min, 95% B.

During each gradient, the flowrates were adjusted as follows; 0-5 min, 1.5 ml/min, 5-6 min, 1.5 ml/min to 1 ml/min; 6-8.5 min, 1 ml/min, 8.5-9 min, 1 ml/min to 1.5 ml/min. The flowrate was reduced to 1 ml/min to increase peak height and improve the limit of detection (peak height a flowrate^{-0.2}) (Snyder, et al., Practical HPLC Method Development, 2nd ed., New York, N.Y.: John Wiley & Sons, p. 653 (1997), which is hereby incorporated by reference in its entirety).

A standard curve over the range 50 to 1000 ng p24^{s} was employed. Analysis was performed on a Alliance 2690 HPLC system (Waters Corporation, Milford, Mass.) with detection at 212 nm using a Waters 996 photodiode array detector. Under these conditions, HBsAg eluted at 7.15 minutes. Western blot analysis of the appropriate fractions confirmed the identity of the peak from plant-derived material.

Detection of p24^{s} by Western blot was performed as follows. Samples were added to buffer (Reducing conditions: 0.1 M Tris pH 8.0, 4% SDS, 1 M DTT final concentrations; Non-reducing conditions: 0.1 M Tris pH 8.0, 2% SDS final concentrations), heated for 20 minutes at 90-95° C., cooled to room temperature and separated by SDS-PAGE (Laemmli, U. K., “Cleavage of Structural Proteins During the Assembly of the Head of Bacteriophage T4,” Nature 227:680-685 (1970), which is hereby incorporated by reference in its entirety) using a 15% polyacrylamide gel. After electrophoretic tank transfer (Towbin, et al., “Electrophoretic Transfer of Proteins From Polyacrylamide Gels to Nitrocellulose Sheets: Procedure and Some Applications,” Proc. Natl. Acad. Sci. USA 76:4350-4354 (1979), which is hereby incorporated by reference in its entirety) to a 0.2 μm polyvinyldenefluoride membrane (BioRad Technologies, Hercules, Calif.) and storage overnight in blocking buffer (1× Dulbecco's PBS, 0.5% Tween-20, 5% dry milk), the blot was probed with goat anti-HBsAg (dilution 1:2000, Fitzgerald, Concord, Mass.), followed by rabbit anti-goat conjugated to horse-radish peroxidase (dilution 1:18000, Sigma, St. Louis, Mo.). The ECL+chemiluminescent kit (Amersham Pharmacia Biotech, Buckinghamshire, England) in conjunction with a Storm 840 phosphorimager (Molecular Dynamics, Sunnyvale, Calif.) were used for image capture. Band intensity increased linearly up to 20 ng of p24^{s}.

### Example 4

### Measurement of Antigenically Reactive HBsAg

Monoclonal and polyclonal ELISAs were used to quantify membrane-associated, antigenically reactive HBsAg. Neither assay will detect free monomer. The Auszyme monoclonal diagnostic kit (Abbott Laboratories, Abbott Park, Ill.) was employed following the manufacturer's instructions, with sample incubation for 16 hours at room temperature (22° C.). The polyclonal sandwich ELISA was performed as described (Dogan, et al., “Process Options in Hepatitis B Surface Antigen Extraction From Transgenic Potato,” Biotechnol. Prog 16:435-441 (2000), which is hereby incorporated by reference in its entirety) with the following modifications: sheep anti-HBsAg (The Binding Site Inc., San Diego, Calif.) capture antibody 1:140 dilution; rabbit anti-α-HBsAg (Accurate Scientific, Westbury, N.Y.) primary antibody 1:600 dilution; anti-α-rabbit horseradish peroxidase conjugate (Sigma Inc., St. Louis, Mo.) 1:12000 dilution; after 4-6 minutes at room temperature for color development, the reaction was stopped using 1 N H₃PO₄. For all, yeast-derived HBsAg (Rhein Biotech, Düsseldorf, Germany) was employed as a standard. In subsequent sections, the following nomenclature will be employed; MAb-HBsAg refers to plant-derived antigen reactive with the Auszyme monoclonal kit; PAb-HBsAg refers to plant-derived antigen detectable by the polyclonal plate ELISA. Total HBsAg protein, i.e. p24^{s}, will include both antigenically reactive and unreactive material.

### Example 5

### Effect of Detergent on HBsAg Antigenicity and Ttotal p24^{s} Extraction

Optimization of Detergent Concentration: The parameter of interest for use in these studies was R, the ratio of the final detergent concentration to the plant material concentration in the lysate. This ratio was varied by adjusting the Triton X-100 (% v/v) concentration in the buffer while maintaining cell mass constant and vice versa. Sodium ascorbate levels were maintained between 1.5 and 2% (w/v). In certain experiments, PBS was replaced by a carbonate/bicarbonate buffer, pH 11, to assess the extent of endoplasmic reticulum breakage (Fujiki, et al., “Isolation of Intracellular Membranes by Means of Sodium Carbonate Treatment: Application to Endoplasmic Reticulum,” J. Cell. Biol. 93:97-102 (1982), which is hereby incorporated by reference in its entirety).

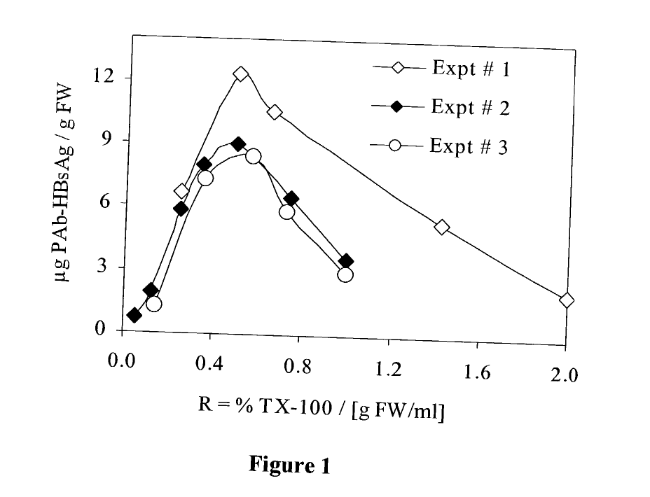

Effect of Detergent on HBsAg Antigenicity: Due to the membrane associated nature of p24^{s}, the effect of detergent level on HBsAg detection was investigated. The ratio of final detergent concentration (% v/v) to plant material concentration (g fresh weight [FW]/ml) (R) was varied from 0 to 4. For transgenic tobacco cell extracts, PAb-HBsAg initially increased, rising approximately 10 fold, with maximum levels measured at an R ratio of 0.5 to 0.6 (FIG. 1). PAb-HBsAg subsequently dropped and, above a ratio of 2.0, was undetectable in the crude lysates.

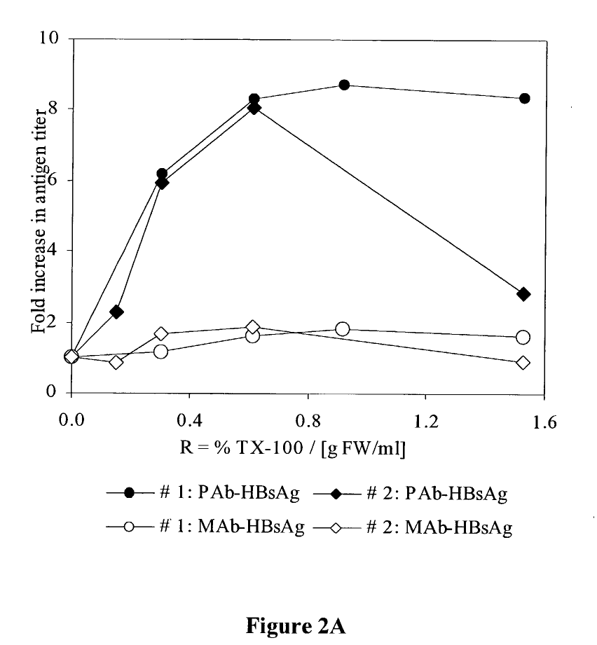

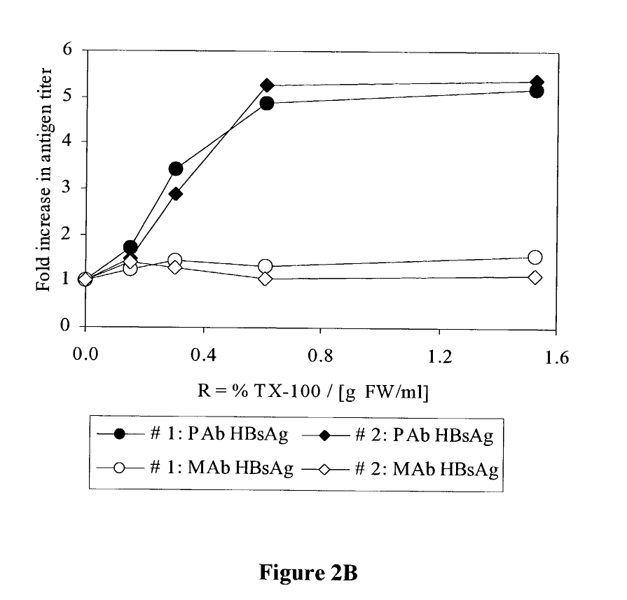

Examining samples which had lost antigenicity (R>2.0) by Western blot showed that the antigen still migrated as a single, well defined band. The yeast-derived HBsAg standard was also tested over the same detergent concentration range and found to be stable under all conditions. These initial studies were extended to include the soybean culture-derived antigen and evaluate the effect of detergent on MAb-HBsAg (FIG. 2). The PAb-HBsAg profiles were similar for both cell lines, with levels rising 5 to 9 fold with increasing R value. At higher R values (>0.6), antigen levels either plateau or fall as previously seen (FIG. 1). The observed changes in MAb-HBsAg were less dramatic; both extracts showed at maximum a 1.5 to 2-fold increase over the detergent range analyzed. Since the maximum PAb-HBsAg titers detected in both cultures differed by 7 fold (45 μg/g FW for soybean cells vs. 6.5 μg/g FW for tobacco cells), the observed profiles were independent of intracellular antigen titers.

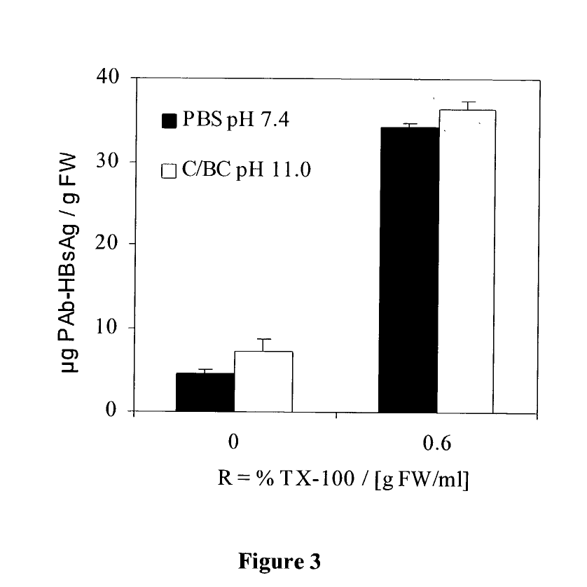

When no detergent was present, the ratio of MAb- to PAb-HBsAg was close to unity; for soybean cell extracts, it varied from 0.9-1.2 and for tobacco cell extracts from 0.7-1.2. One possible function of the detergent would be to aid in the release of particles that are encased within the membrane of the endoplasmic reticulum (ER). To test this possibility, extraction was performed using either the standard PBS-based buffer (pH 7.4) or a sodium carbonate buffer (pH 11.0), both in the absence of detergent. The low salt, high pH conditions of the carbonate buffer converts vesicles into sheets, releasing the ER lumen contents, while leaving integral membrane proteins, associated with the ER, intact (Fujiki, et al., “Isolation of Intracellular Membranes by Means of Sodium Carbonate Treatment: Application to Endoplasmic Reticulum,” J. Cell. Biol. 93:97-102 (1982), which is hereby incorporated by reference in its entirety). Previous studies have shown that HBsAg particles are unaffected by this procedure (Simon, et al., “A Block to the Intracellular Transport and Assembly of Hepatitis B Surface Antigen Polypeptides in Xenopus ooctyes,” Virology 166:76-81 (1988), which is hereby incorporated by reference in its entirety). When carbonate replaced the PBS-buffer, the detectable PAb-HBsAg rose by 50%, indicating improved HBsAg release; however, the addition of detergent led to a 5-fold rise in detectable PAb-HBsAg titers to levels identical to those obtained by extraction with the PBS/Triton X-100 buffer (FIG. 3). Therefore, even with all the ER lumenal content released, the majority of the PAb-HBsAg was not in a form detectable by the immunoassay. A similar profile for PAb-HBsAg versus R was obtained for potato HB114-16 tuber extracts; an R ratio of 0.6 yielded the maximum detectable antigen, with titers falling as detergent level was further increased.

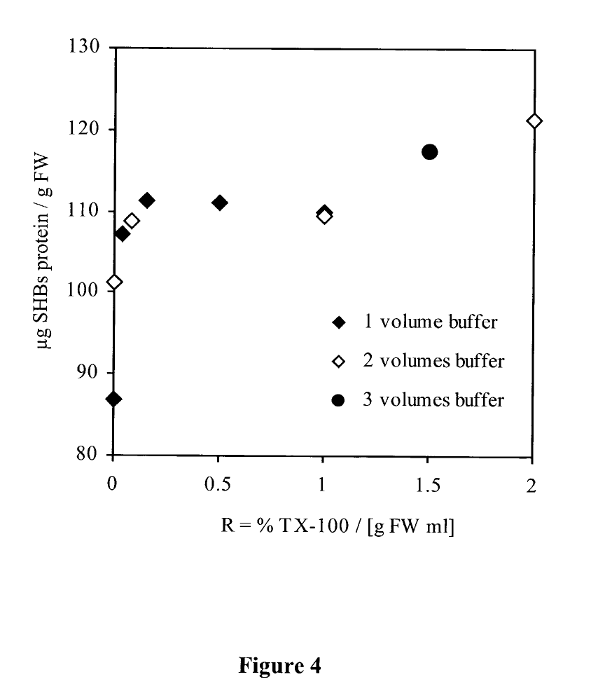

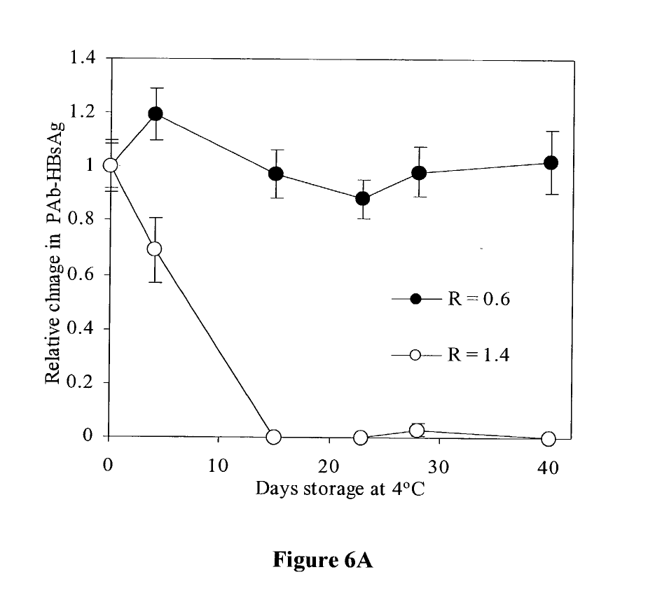

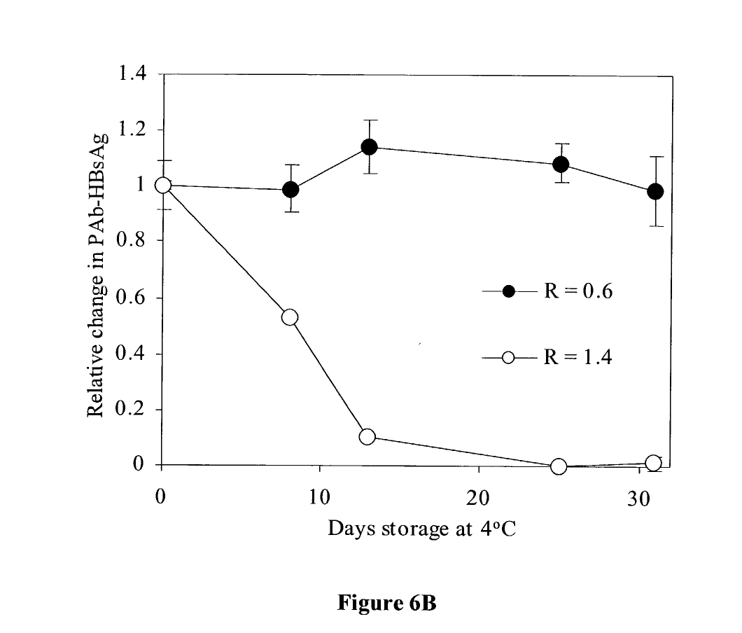

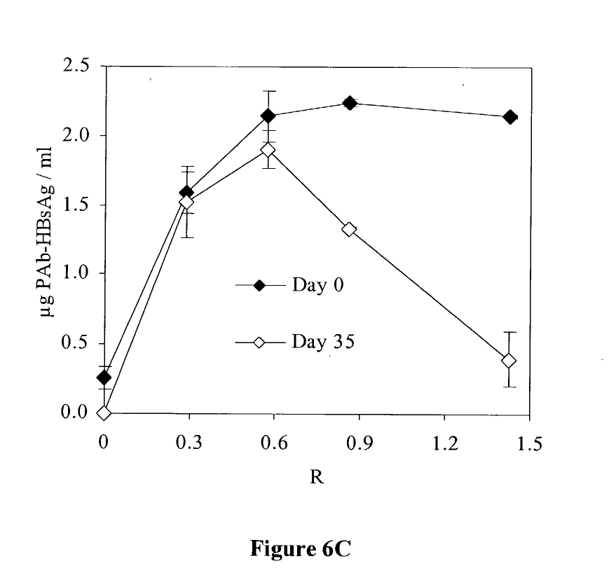

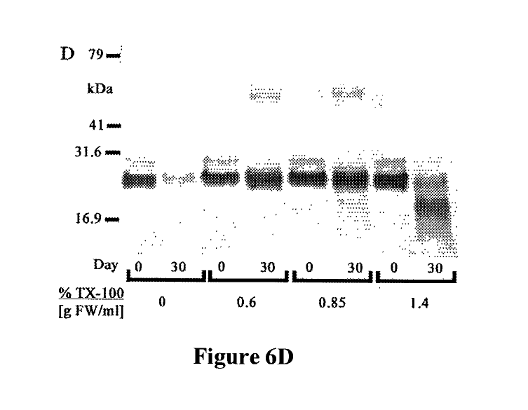

To assess the effect of detergent on total p24^{s} extraction, crude cell lysates obtained over a range of R values were analyzed by RP-HPLC (FIG. 4). Even in the absence of detergent, p24^{s} was efficiently extracted; the addition of detergent improved release from the cell debris by only 8-20%, depending on buffer volume to cell ratio, and above an R value of 0.1, p24^{s} titers plateau/increase gradually. To confirm that all antigen was recovered from the cells, the lysis step was omitted and the tissue culture material solubilized directly in the HPLC pre-treatment buffer. This yielded a p24^{s} concentration of 112+/−4 μg/g FW, in very good agreement with the maximal levels previously obtained. Total p24^{s} in extracts obtained by Fastprep lysis over a range of R values was also analyzed by Western blot (FIG. 6D, Day 0 lanes). Increasing R from 0 to 1.6 resulted in a 1.5 fold increase in p24^{s} levels. These results demonstrate that the extraction of p24^{s} is relatively insensitive to R value. The substantial increase in PAb-HBsAg profiles observed (FIGS. 1 and 2) was principally due to improved detection of extracted HBsAg resulting from the increased levels of detergent.

The primary effect of detergent was the conversion of already extracted HBsAg into a form detectable by the PAb-immunoassay. The addition of increasing levels of detergent produced a 5 to 8 fold increase in PAb-HBsAg (FIG. 2) but only a modest improvement in total p24^{s} extracted from the plant cells (FIGS. 4 & 7D). Since HBsAg VLPs are created by the inward budding of p24^{s}-decorated ER membrane (Eble, et al., “Hepatitis B Surface Antigen: An Unusual Secreted Protein Initially Synthesized As A Transmembrane Polypeptide,” Mol. Cell. Biol. 6:1454-1463 (1986), which is hereby incorporated by reference in its entirety), the detergent could function by permeabilizing this membrane, releasing the lumenal contents. Carbonate extraction, which converts closed vesicles into open sheets (Fujiki, et al., “Isolation of Intracellular Membranes by Means of Sodium Carbonate Treatment: Application to Endoplasmic Reticulum,” J. Cell. Biol. 93:97-102 (1982), which is hereby incorporated by reference in its entirety), did not increase PAb-HBsAg appreciably, indicating this was not the detergent's principal mechanism of action. This observation also suggests that the majority of p24^{s} was not in a particulate form, but it remains associated with the ER membrane. Triton X-100 can promote vesicle formation by the inward budding of ER membrane (Kreibich, et al., “Recovery of Ribophorins and Ribosomes in ‘Inverted Rough’ Vesicles Derived From Rat Liver Microsomes,” J. Cell. Biol. 93:111-121 (1982), which is hereby incorporated by reference in its entirety), and the conversion of ER membrane into VLPs appears to be the process by which the PAb-HBsAg is converted to a detectable form. Sucrose gradient analysis of cell lysates under different detergent conditions supports this mechanism (see Example 10).

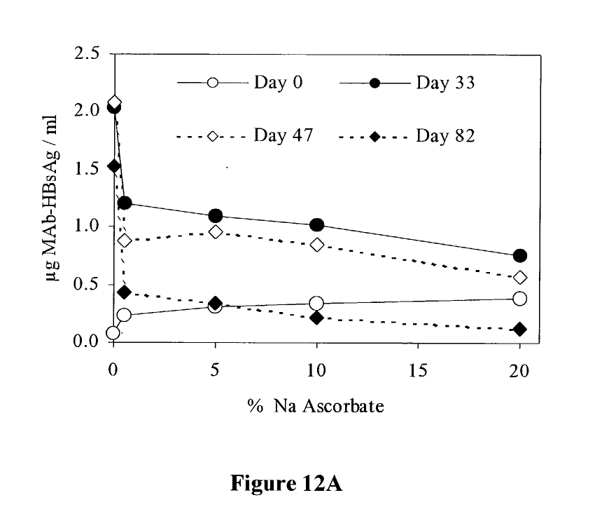

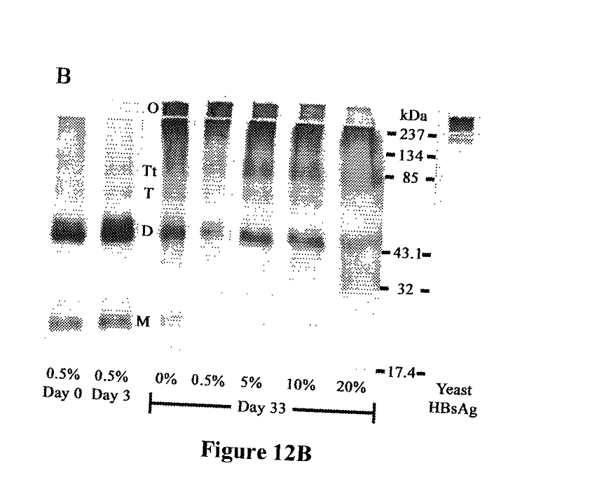

When levels of detergent were raised further, PAb-HBsAg titers either plateaued or decreased. Interestingly the optimum ratio of detergent to plant cell material was similar for both the soybean and tobacco extracts, although HBsAg titers were markedly different (FIGS. 1 and 2). The consistency of this ratio suggests that the total lipid content of the extract and not HBsAg titers determines the required detergent concentration. The variability observed at higher ratios may result from different total lipid content of the samples used. When the yeast standard was spiked into buffers over the same detergent range its PAb-reactivity was unaffected. The higher susceptibility of the plant-derived antigen to detergent may result from differences in disulfide bonding, the yeast-derived standard being fully cross-linked compared to the plant HBsAg, which existed predominantly as dimers (FIG. 12B). Extensive cross-linking imparts stability to the VLPs; reduction of cross-linked, serum-derived HBsAg prior to treatment with 1% non-ionic detergent compromised the antigens particulate structure (Galivanes, et al., “Hepatitis B Surface Antigen: Role of Lipids in Maintaining the Structural and Antigenic Properties of Protein Components,” Biochem. J. 265:857-864 (1990), which is hereby incorporated by reference in its entirety).

In contrast to PAb-HBsAg, the titers of MAb-HBsAg were unaffected by increasing detergent (FIG. 2), suggesting that the majority of the antigen displaying the MAb-epitopes resided on the readily detectable VLPs released from the ER lumen. The particle structure may be necessary for the formation of these epitopes, which would explain their absence on the detergent-generated VLPs formed from ER membrane. Similar profiles were obtained for potato tuber and tomato fruit extracts, indicating a similar HBsAg distribution to the plant cell culture material.

### Example 6

### Influence of Sodium Ascorbate on HBsAg Antigenicity

Sample preparation and conditions: The concentration of the anti-oxidant sodium ascorbate (Na Asc) was varied from 0 to 20% (w/v) in the extraction buffer. Over this range, the pH of the PBS buffer did not change; pH of the 20% (w/v) Na Asc was 7.4. For experiments to assess the effect of atmospheric oxygen on plant-derived HBsAg, extraction buffer that had been purged for 1 minute with nitrogen was used, and the buffer added to the sample tube under a nitrogen environment. The detergent to cell concentration ratio (R) was maintained at 0.6.

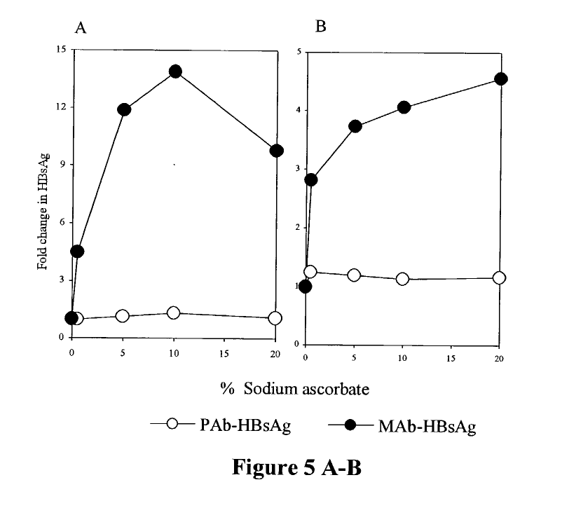

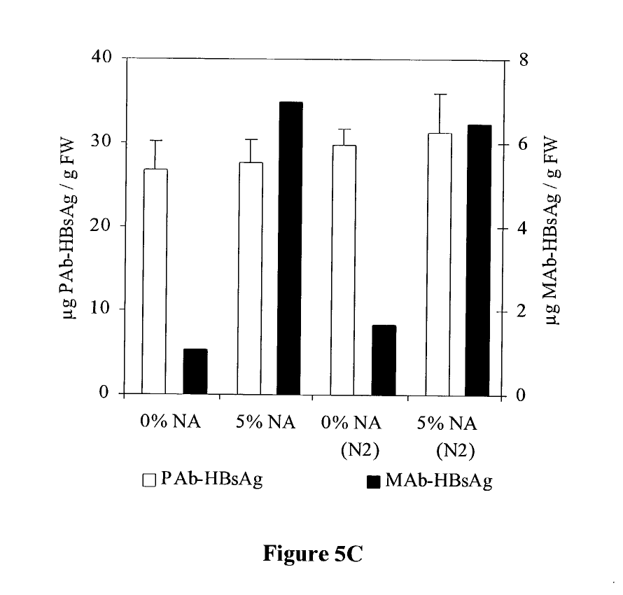

Influence of Sodium Ascorbate and Atmospheric Oxygen on HBsAg Antigenicity: Sodium ascorbate was the other component of the extraction buffer tested (FIGS. 5A and B). This antioxidant had no effect on PAb-HBsAg detected; however, it substantially increased the amount of antigen reactive with the monoclonal assay; a 14-fold and a 4.6-fold increase was observed in crude extracts of transgenic tobacco and soybean suspension cells. At their maximum, the MAb-HBsAg represented 12% and 24% of PAb-HBsAg for soybean and tobacco, respectively.

Extraction with a hand-held pestle was performed in parallel in an attempt to minimize sample oxidation and determine if the influence of the sodium ascorbate could be reduced. The beneficial effect of sodium ascorbate was similar to the Fastprep extracted samples. Finally, extraction under a nitrogen environment was tested (FIG. 5C); although this resulted in a 1.5 fold increase in MAb-HBsAg levels when no ascorbate was present, the addition of the antioxidant still resulted in a dramatic increase in MAb-HBsAg titers, to a level identical to when extraction was performed in the absence of nitrogen. In contrast, sodium ascorbate had no effect on MAb-HBsAg in potato tuber crude extracts, which represented 10% of PAb-HBsAg over the full sodium ascorbate range tested (0-20% w/v).

### Example 7

### Influence of Detergent and Sodium Ascorbate on HBsAg Stability

Optimization of Detergent Concentration: The parameter of interest in for use in these studies was R, the ratio of the final detergent concentration to the plant material concentration in the lysate. This ratio was varied by adjusting the Triton X-100 (% v/v) concentration in the buffer while maintaining cell mass constant and vice versa. Sodium ascorbate levels were maintained between 1.5 and 2% (w/v). In certain experiments, PBS was replaced by a carbonate/bicarbonate buffer, pH 11.

Influence of Detergent and Sodium Ascorbate on HBsAg Stability: Initial observations indicated that HBsAg was unstable in crude extracts from both tobacco and soybean suspension cultures, with a complete loss of PAb-HBsAg after 15-20 days of storage at 4° C. (FIGS. 6A-B). Lowering the detergent to cell concentration ratio (R) from the initially employed level of 1.4 to 0.6 dramatically improved antigen stability, with PAb-HBsAg levels stable for 30-40 days with storage at 4° C. (FIGS. 6A-B). Stability over a range of R values was, therefore, tested. For soybean crude lysates, PAb-HBsAg levels remained stable for R between 0.3 and 0.6, but dropped at higher values or when detergent was omitted (FIG. 6C).

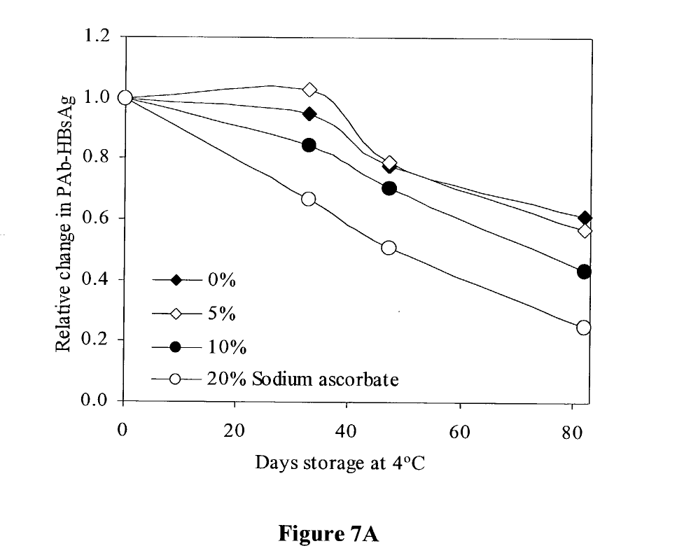

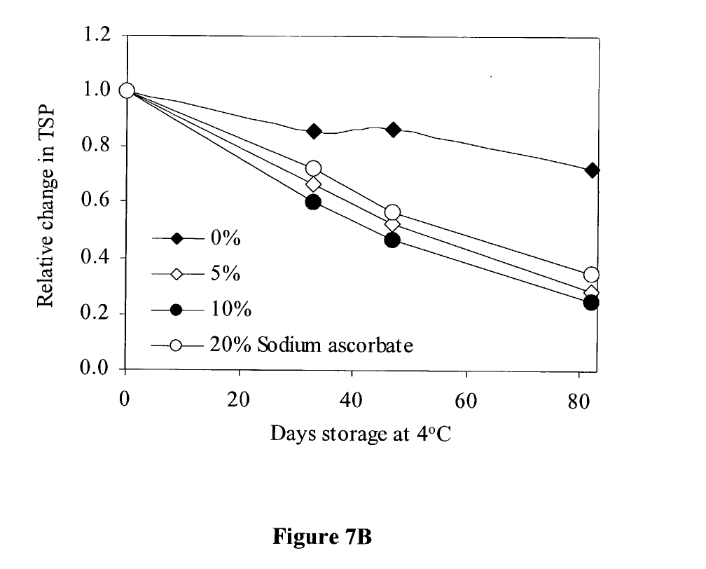

Comparing the fresh and aged extracts by reducing Western blot demonstrated that the loss at R=0 was due to precipitation, whereas at R=1.4 proteolytic degradation had occurred (FIG. 6D). The precipitation in the absence of detergent was not always observed; in these cases, the PAb-HBsAg was stable in solution. For the stable samples, there was, however, a loss of the 27 kDa band with storage. A similar PAb-HBsAg profile was observed for tobacco cell extracts tested over the same range of R values. Over an extended time-scale a loss in PAb-HBsAg was observed with storage (FIG. 7A). This loss was exacerbated by the presence of elevated levels of sodium ascorbate which also increased the rate of total protein loss from solution (FIG. 7B).

Measurement of Total Soluble Protein: The present assay was employed to assess the influence of sodium ascorbate on total soluble protein levels in crude extracts with storage. Total soluble protein was measured by the method of Bradford (Bradford, M. M., “A Rapid and Sensitive Method for the Quantitation of Microgram Quantities of Proteins Utilizing the Principle of Protein-Dye Binding,” Anal. Biochem. 72:248-254 (1976), which is hereby incorporated by reference in its entirety) using the BioRad Protein Reagent (BioRad Laboratories, Hercules, Calif.) following the accompanying microtiter plate protocol. The absorbance was read at 595 nm and 450 nm in a microplate reader (Dynex Technologies, Chantilly, Va.), and the ratio of the absorbances, 595 nm over 450 nm, was used for standard curve calculations (Zor, et al., “Linearization of the Bradford Protein Assay Increases Its Sensitivity: Theoretical and Experimental Studies,” Anal. Biochem. 236:302-308 (1996), which is hereby incorporated by reference in its entirety). Bovine Serum Albumin (Pierce, Rockford, Ill.) was employed as a standard.

The stability of a recombinant protein will depend both on the protein itself and the plant background in which it is expressed. An IgG-monoclonal antibody (Khoudi, et al., “Production of a Diagnostic Monoclonal Antibody in Perennial Alfalfa Plants,” Biotechnol. Bioeng. 64:135-143 (1999), which is hereby incorporated by reference in its entirety) was found to be stable for 4 days in alfalfa juice, while it was precipitated when spiked into tobacco leaf extract. In addition, both extracellular and intracellular protein degradation have recently been shown to compromise the integrity of plant-produced antibodies (Sharp, et al., “Characterization of Monoclonal Antibody Fragments Produced in Plant Cells,” Biotechnol. Bioeng. 73:338-346 (2001), which is hereby incorporated by reference in its entirety). For orally delivered plant vaccines, some processing will be required to ensure consistent antigen dosing. The subsequent loss in compartmentalization could expose the antigen to proteases (Gegenheimer, P., “Preparation of Extracts From Plants,” Meth. Enzymol. 182:174-194 (1990), which is hereby incorporated by reference in its entirety), polyphenol oxidases and plant phenolics (Loomis, W. D., “Overcoming Problems of Phenolics and Quinones in the Isolation of Plant Enzymes and Organelles,” Meth. Enzamol. 31:528-544 (1974), which is hereby incorporated by reference in its entirety), compromising its immunogenic epitopes. Serum-derived HBsAg has been shown to be remarkably protease resistant, a characteristic attributable to its extensive cross-linking (Peterson, D. L., “Isolation and Characterization of the Major Protein and Glycoprotein of Hepatitis B Surface Antigen,” J. Biol. Chem. 256:6975-6983 (1981), which is hereby incorporated by reference in its entirety) which is lacking in the plant-derived antigen (FIG. 12B). Consequently, HBsAg was susceptible to proteolysis in both soybean and tobacco cell crude lysates (FIG. 6D). However, by optimizing the ratio of detergent to cell concentration, the antigen could be stabilized during storage for up to one month. This excellent stability resulted from the membrane-associated nature of p24^{s}, with excessive detergent compromising lipid bilayer masking of protease-sensitive regions. Loss of the minor 27 kDa band, which likely represented glycosylated p24^{s} (Peterson, D. L., “Isolation and Characterization of the Major Protein and Glycoprotein of Hepatitis B Surface Antigen,” J. Biol. Chem. 256:6975-6983 (1981), which is hereby incorporated by reference in its entirety), did occur, suggesting that the antigen was still susceptible to endoglycosidase activity. For HBsAg, this is not of concern, since the glycan is not required for immunogenicity (Gerlich, et al., “Functions of Hepatitis B Virus Proteins and Molecular Targets for Protective Immunity,” R. W. Ellis, Ed., In Hepatitis B Vaccines in Clinical Practice, New York, N.Y.: Marcel Dekker, pp. 41-82 (1993), which is hereby incorporated by reference in its entirety). However, for storage times in excess of one month, a gradual loss in PAb-HBsAg was observed. This loss was exacerbated by the presence of sodium ascorbate which also accelerated total protein precipitation (FIG. 7); increased precipitate formation with sodium ascorbate was observed visually in the stored extracts. The mechanism by which sodium ascorbate enhanced precipitation was not clear.

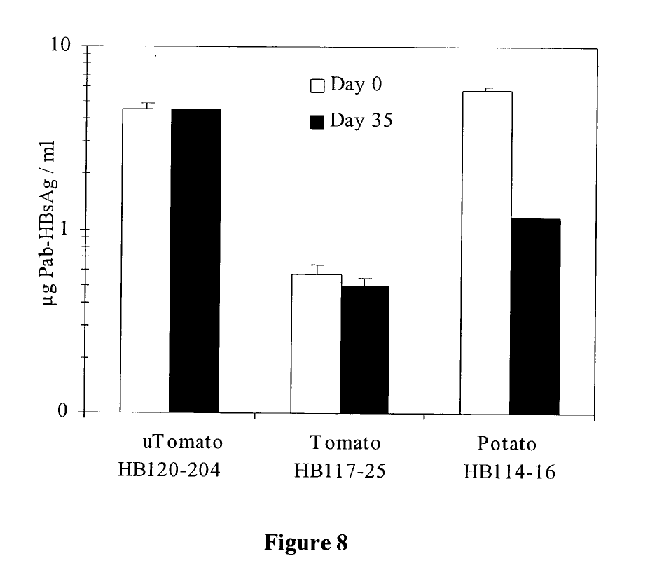

Potato tuber-derived PAb-HBsAg was unstable with storage of the crude lysates. Even at the optimal R ratio, PAb-HBsAg titers fell approximately 5 fold following 1 month of storage at 4° C. (FIG. 8). In contrast, for tomato extracts (either from micro-μ-Tom HB120-204 or Tomato HB117-25) at an optimal R value of 0.6, PAb-HBsAg levels were stable for at least 35 days with storage at 4° C. (FIG. 8). Examples 8 and 9 both discuss stabilization by milk and component with some data repetition. They have been combined. In vitro assembly is not discussed. See Example 9.

### Example 8

### Stability of Plant-Derived HBsAg With Storage and Effect of Skim Milk on HBsAg Stability

The excipients tested for their influence on HBsAg stability in crude lysates were glycerol, dry skim milk (Difco, Sparks, Md.), lactose, whey (Sigma, St. Louis, Mo.), whey protein concentrate (Provon® 290, Avonmore Waterford Ingredients Inc., Monroe, Wis.) and casein (sodium caseinate F&P, American Casein Company, Burlington, N.J.). The whey (Sigma) was a spray-dried powder containing 11% protein (minimum) and approximately 65% lactose. For the stability studies, 750 μl of cleared cell lysate was combined with 250 μl of a skim milk solution (dissolved in PBS and adjusted to pH 7.4) and stored at 4° C. Final skim milk concentrations in the range of 0.5% to 20% w/v were tested. For storage in the presence of the constituents of dry skim milk, 8 g cell samples was combined with 40 ml buffer and extracted using a Waring Blender (Waring Commercial, New Hartford, Conn.). The cleared lysate (300 μl) was combined with 700 μl excipient (dissolved in PBS and adjusted to pH 7.4). Samples were assayed initially and after approximately 1 month storage at 4° C. PBS alone was used as a control. Depending on the component, concentrations in the range of 0.5% to 35% were tested.

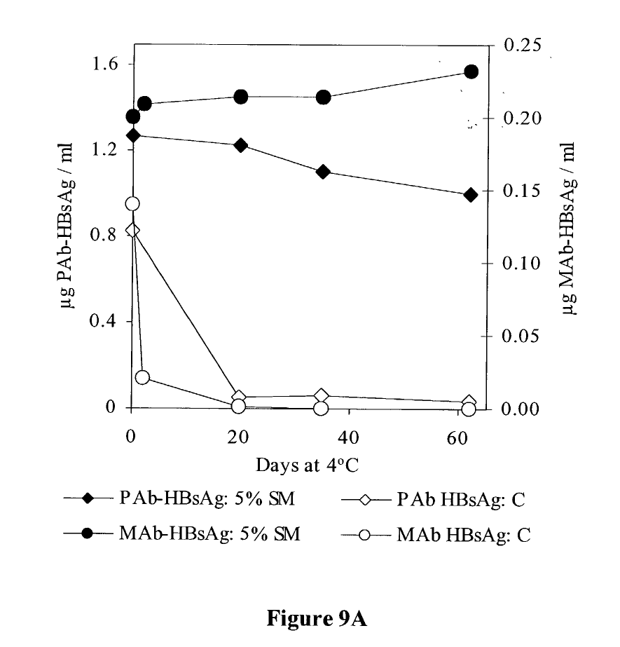

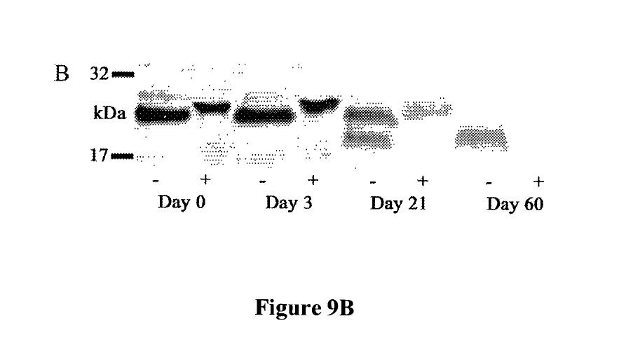

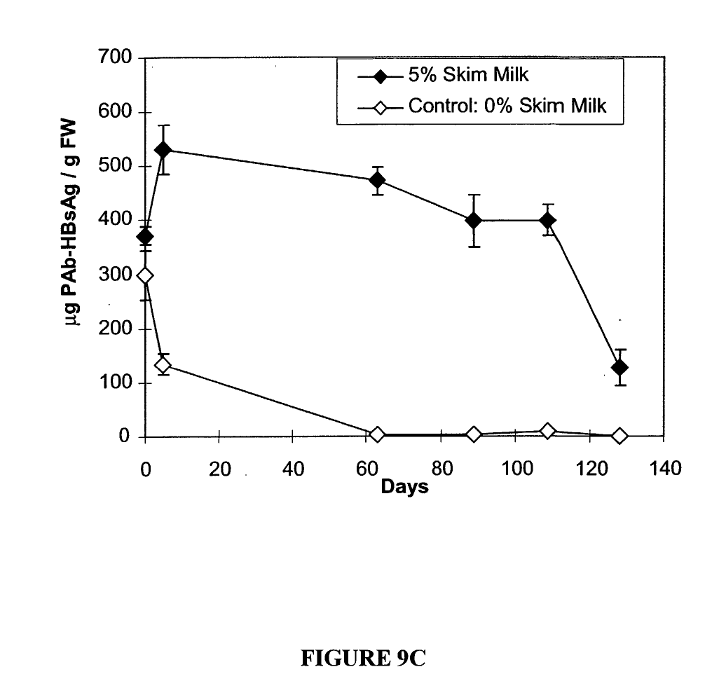

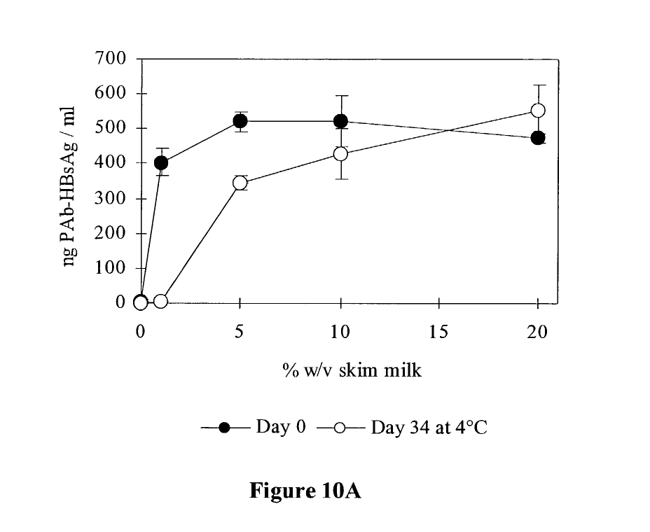

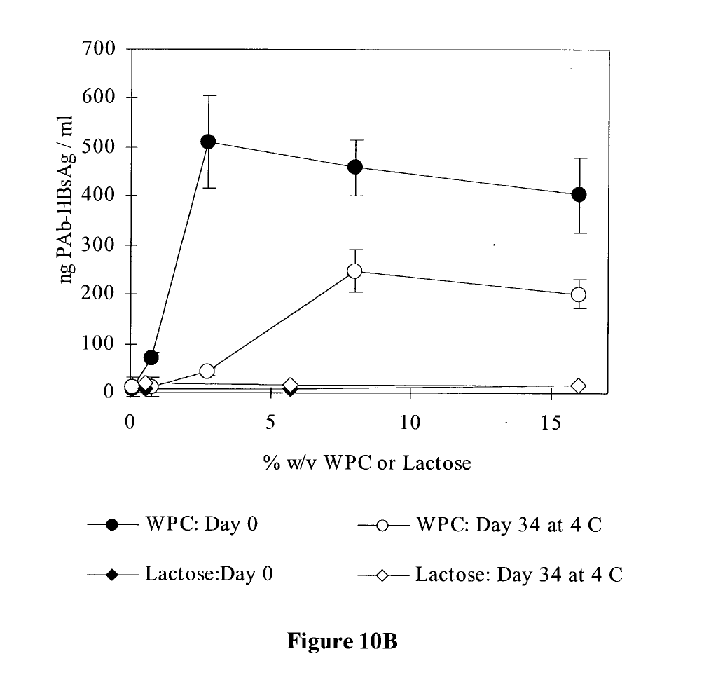

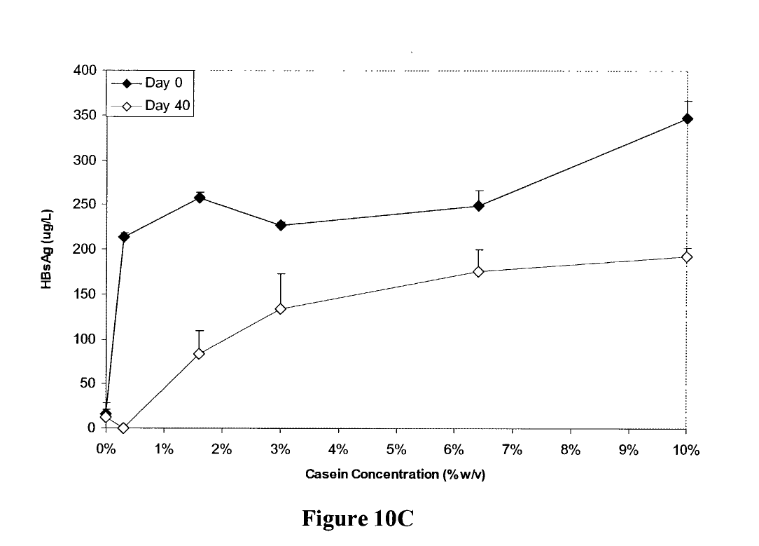

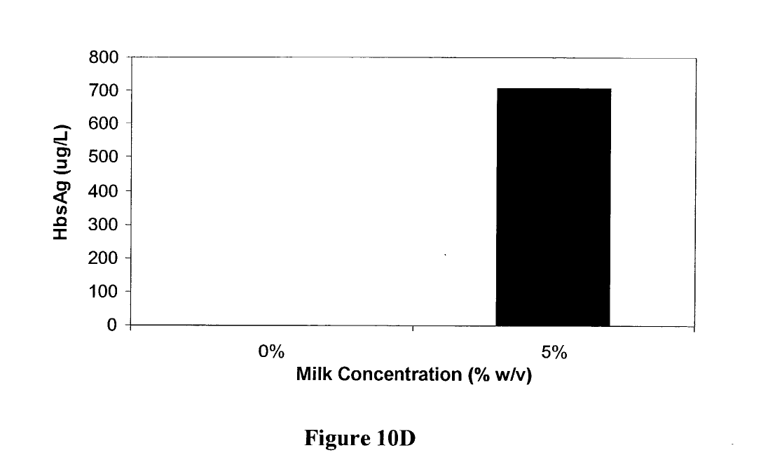

In the present invention, different milk components were found to have different stabilizing effects, when used in different proportions or amounts. Results are summarized graphically in FIGS. 9 and 10 and in tabular form in Tables 2 and 3. In most instances, the protein component of milk was found to be responsible in part for the stabilizing effect of skim milk on HBsAg in crude protein extracts, but is not as effective as complete skim milk.

Greater product storage stability under ambient and/or normal use environments is observed with the stabilized hepatitis B surface antigen plant cell suspension(s), plant extract(s) (i.e, after stabilization treatment of HBsAg protein via methods of the present invention). For example, Tables 2 and 3 shows the effect of skim milk derivatives on HBsAg levels measured in crude protein extracts after extraction (Day 0) and after storage for approximately one month at 4° C. (Day 34). In addition, FIGS. 9 and 10 illustrate routine experimental long term storage and stability studies, which examine different samples of HBsAg as expressed in different plant cell suspensions, plant extracts, etc., stabilization with varying amounts and/or concentrations of different milk protein and/or corresponding milk components thereof, extraction buffers, and environmental effects, such as temperature and time, as expressed in number of days or months.

TABLE 2
Effect of Skim Milk and Skim Milk Derivatives on
PAb-HBsAg Levels Measured in Crude Protein Extracts of
Tobacco NT1 HB155-18 Suspension Cells After Extraction
(Day 0) and After Storage for Approximately One Month
at 4° C. (Day 34)

| | Day 0 | Day 34 |
|---|---|---|
| Skim Milk | | |
| 0% | 22 +/− 4 | 7 +/− 10 |
| 1% | 1237 +/− 118 | 11 +/− 13 |
| 5% | 1593 +/− 86 | 1054 +/− 59 |
| 10% | 1596 +/− 26 | 1309 +/− 217 |
| 20% | 1446 +/− 40 | 1695 +/− 492 |
| 35% | 1245 +/− 167 | 1268 +/− 331 |
| Whey | | |
| 0% | 22 +/− 4 | 7 +/− 10 |
| 0.4% | 344 +/− 41 | 4 +/− 4 |
| 4% | 1410 +/− 223 | 710 +/− 112 |
| 13% | 1436 +/− 406 | 1824 +/− 276 |
| 35% | 1080 +/− 298 | 431 +/− 36 |
| Whey Protein concentrate | | |
| 0% | 22 +/− 4 | 7 +/− 10 |
| 0.08% | 25 +/− 8 | 38 |
| 1% | 220 +/− 27 | 36 |
| 2.7% | 1567 +/− 290 | 132 |
| 8% | 1403 +/− 172 | 759 |
| 16% | 1233 +/− 238 | 619 |
| Lactose | | |
| 0% | 22 +/− 4 | 7 +/− 10 |
| 1% | 24 +/− 18 | 57 +/− 54 |
| 6% | 23 +/− 13 | 48 +/− 50 |
| 20% | 51 +/− 11 | 47 +/− 7 |

In particular, the hepatitis B surface antigen in crude cell suspension or extracts of the present invention may be stabilized by the addition of skim milk, at a final concentrations of 5% (w/v) and retained antigenicity for greater than 100 days, at a final concentrations of 5% (w/v) (FIG. 9C). In extracts lacking skim milk, HBsAg antigenicity dropped rapidly and was no longer detectable after 60 days. The protein component of the milk was responsible for the stabilization, though the protein components alone were not as effective at maintaining antigenicity as skim milk. See FIG. 9A, which shows the effect of the addition of skim milk (final concentration 5% w/v) on HBsAg stability in crude transgenic soybean W82 HB155-37 cell lysates with storage at 4° C. and FIG. 9B, which shows a Western blot (reducing conditions) of select samples at various ages.

Effect of Skim Milk on HBsAg Stability: At a R ratio 1.4, several avenues to increasing the stability of HbsAg in crude plant extracts were tested. Initially, several protease inhibitors were included in the extraction buffer. Preliminary results indicated that 1 mM phenylmethylsulfonyl fluoride (PMSF) improved short-term stability (<5 days); however, at later times, antigen loss was comparable to controls.

An extensive evaluation of all the protease inhibitor classes was not performed, as the majority are toxic to humans and their use was, therefore, contrary to the goal of an oral vaccine formulation produced using minimal downstream processing.

Other “edible” stabilizers were, therefore, tested, namely glycerol and dry skim milk. Glycerol, at 10-20% (v/v), is often used to stabilize enzymes in solution (Deutscher, M. P., “Maintaining Protein Stability,” Meth. Enzymol. 182:83-93 (1990), which is hereby incorporated by reference in its entirety) but provided no benefit in the case of tissue-culture derived HBsAg. The protein concentration in solution also affects stability, with higher concentrations (>1 mg/ml) being preferable (Deutscher, M. P., “Maintaining Protein Stability,” Meth. Enzymol. 182:83-93 (1990), which is hereby incorporated by reference in its entirety).

Addition of skim milk powder (5% w/v) increased total protein concentration 43 fold to 10 mg/ml, which effectively stabilized the antigen with storage at 4° C. Both PAb- and MAb-HBsAg were maintained at initial levels for at least 60 days in the case of soybean HB155-37 crude lysates, whereas, in extracts lacking skim milk, PAb-HBsAg was lost within 20 days and MAb HBsAg within three days (FIG. 9A). Similar results were obtained for tobacco cell lysates where stability of the PAb-HBsAg was maintained at initial levels for 90 days.

To determine the mechanism of protection afforded by skim milk, samples of the soybean crude lysate were analyzed by reducing Western blot (FIG. 9B). In the absence of milk, proteolytic degradation was evident by day 21, and, by day 60, all p24^{s} had been reduced to a diffuse band corresponding to a 19-21 kb fragment. No degradation occurred in the presence of skim milk though a reduction in band intensity with storage indicated p24^{s} precipitation.

Skim milk is a complex mixture of proteins (casein and whey protein), lactose and minerals (Ensminger, et al., Foods and Nutrition Encyclopedia, Vol. 1, 2nd ed., Boca Raton, Fla.: CRC Press, pp 980, 988 (1994), which is hereby incorporated by reference in its entirety). To confirm that the protein component of milk was responsible for the conferred stability, crude protein extracts of tobacco cells were combined with varying concentrations of either skim milk or its major components (excluding minerals). Initial antigenicity (˜4 hours after extraction) and antigenicity after 34 days (storage at 4° C.) were compared (FIGS. 10A-B).

For skim milk, initial PAb-HBsAg level was concentration dependent and plateaued at 5% (w/v) (FIG. 10A). No PAb-HBsAg was detectable in the initial control samples (no excipients) in contrast with the profile in FIG. 9A. However, a Waring blender was employed for extraction in this case and not the Fastprep cell disruptor. The protection afforded by the skim milk with storage was also concentration dependent up to 10% w/v skim dry milk and above this the initial PAb-HBsAg titer was maintained. For the skim-milk components (FIG. 10B), lactose afforded no initial protection at any of the concentrations tested, indicating that the carbohydrate component of milk alone provided no benefit. The whey protein concentrate (WPC), at concentrations of approximately 3% to 16%, was initially as effective as skim milk. However, the protection afforded with storage was lower with final PAB-HBsAg levels being 50-55% of the starting values. The profile and degree of protection afforded by casein was similar to WPC, showing the protein component of skim milk was responsible for HBsAg protection. Table 3 shows the effect of casein on HBsAg levels measured in crude protein extracts after extraction (Day 0) and after storage for approximately 40 days at 4° C.

TABLE 3
Effect of Casein on HBsAg Levels

| Casein | Day 0 | Day 40 |
|---|---|---|
| 0% | 16 +/− 5 | 12 +/− 17 |
| 0.3% | 213 +/− 5 | 0 +/− 0 |
| 1.6% | 258 +/− 6 | 83 +/− 27 |
| 3% | 227 +/− 4 | 133 +/− 40 |
| 6% | 249 +/− 18 | 175 +/− 24 |
| 10% | 348 +/− 20 | 193 +/− 9 |

The Auszyme diagnostic assay was also employed to measure the stabilizing effect of milk. From the samples in Table 2, the PBS control and one of the 5% milk samples were compared on Day 0. FIG. 10D shows that the presence of skim milk was required to maintain measurable HBsAg. The measured level (700 μg/L) is lower than that of the ELISA (16001 μg/L). This is because the Auszyme assay employs a monoclonal antibody specific for the group specific a determinant, whereas the polyclonal ELISA will detect membrane associated HBsAg. If this epitope is not correctly formed or effected by the processing method, no detection will occur. Without milk, none of the a determinant was detectable. For partially purified HBsAg to be an effective vaccine, it is essential that this epitope be retained. Antibodies against this epitope are required for the generation of an effective immune response against Hepatitis B.

Under unfavorable detergent conditions, it was possible to effectively stabilize HBsAg by augmenting the protein concentration in the extract by the addition of skim milk or one of its protein constituents (FIGS. 9 and 10). The higher protein content prevented proteolytic degradation of p24^{s} presumably by competing for protease, although precipitation of the antigen still occurred (FIG. 9B). In spite of this loss, the levels of antigenic p24^{s} remained stable, suggesting that incorrectly folded p24^{s} was more susceptible to precipitation. Although, under the appropriate detergent conditions, skim milk addition was not required for HBsAg, this strategy may provide a low cost method for preventing proteolysis of non-membrane associated proteins and would avoid the need to employ costly and toxic protease inhibitor cocktails. Under the optimized detergent conditions, the antigen from tomato fruit extracts showed similar stability to that from cell suspensions; however, it was unstable in potato extracts (FIG. 8). Further buffer modification is required in the latter case; such modification will be easier once the mechanism of instability, i.e. precipitation or proteolysis, has been determined.

### Example 9

### In Vitro Formation of the a Determinant Epitopes of HBsAg

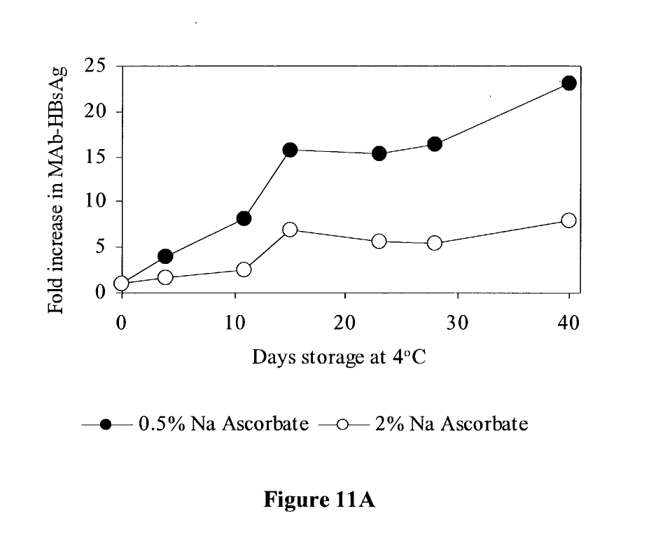

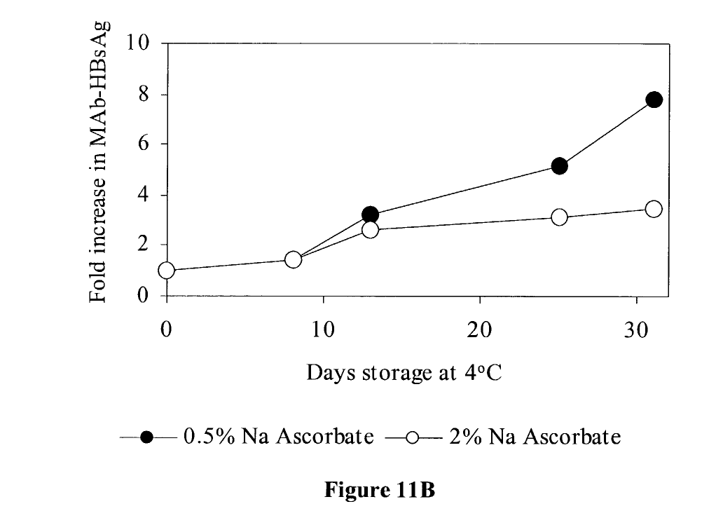

During the course of the stability testing, an increase in MAb-HBsAg was observed with storage at 4° C., for crude lysates of both soybean and tobacco cells (FIG. 11). This increase was influenced by the presence of sodium ascorbate; although the initial level of MAb-HBsAg increased with increasing levels of antioxidant (FIGS. 5A-B), the rate of formation of the MAb-reactive epitopes was retarded by the reducing environment (FIG. 11). In addition, final MAb-HBsAg titers were lower.

Soybean cell extracts were subsequently followed for an 82 day period, with sodium ascorbate levels ranging from 0% to 20% (FIG. 12A). The addition of even low levels of antioxidant (0.5% w/v) had a detrimental effect on MAb-HBsAg formation, with titers after 33 days of storage being 50% lower than the sodium ascorbate free samples.

As the samples were stored over longer periods of time, there was a substantial loss in MAb-HBsAg in samples containing sodium ascorbate; titers were reduced by 65-85%. In contrast, titers fell by only 25% for the extracts lacking sodium ascorbate.

Similar results were obtained for transgenic tobacco extracts, and these observations paralleled those for PAb-HBsAg (FIG. 7). Non-reducing Western blots demonstrated that extensive disulfide bonding occurred during sample storage (FIG. 12B). Initially and after three days storage at 4° C., the antigen was principally present as dimers and monomers, with trimers and tetramers present to a lesser degree.

With extended sample storage (33 days), the monomer and dimer subpopulations were reduced with a concurrent increase in higher order oligomers. A substantial fraction of the aged plant-derived antigen was trapped in the 4% stacking gel, similar to the purified yeast-derived standard.

The levels of higher order oligomers also decreased with increasing antioxidant level. Unlike the plant cell culture extracts, no increase in MAb-HBsAg was observed with storage of potato tuber extracts (35 days at 4° C.); MAb-HBsAg titers either remained constant or declined at the higher (>5% w/v) antioxidant levels. Similarly, for tomato extracts, MAb-HBsAg levels either remained constant (Tomato HB117-25) or rose by approximately 1.5-fold (μTom HB120-204), indicating that the dramatic rise in MAb-HBsAg observed was unique to the plant cell culture extracts.

Altering the buffer and storage conditions will affect the in vitro disulfide bond rearrangements. For yeast-derived HBsAg, increasing the pH above 11 accelerated the formation of higher order oligomers as did increasing the incubation temperature (Wampler, et al., “Multiple Chemical Forms of The Hepatitis B Surface Antigen Produced in Yeast,” Proc. Nat'l Acad. Sci. USA 82:6830-6834 (1985) and U.S. Pat. No. 4,707,542 to Friedman et al., which are hereby incorporated by reference in their entirety).

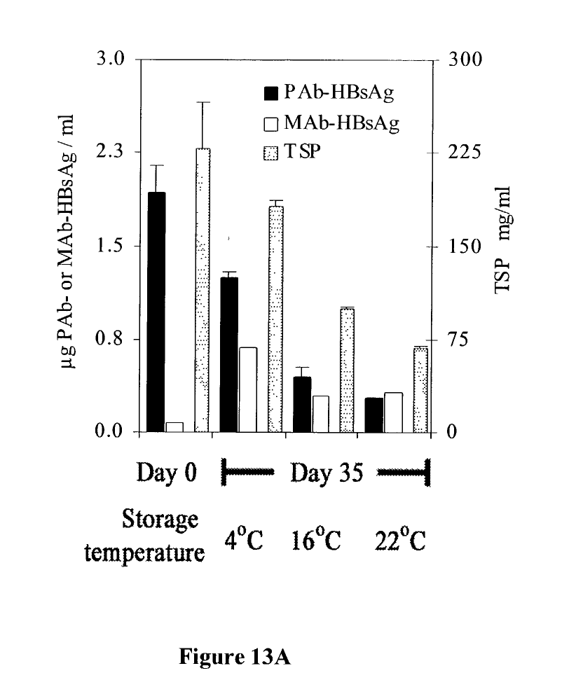

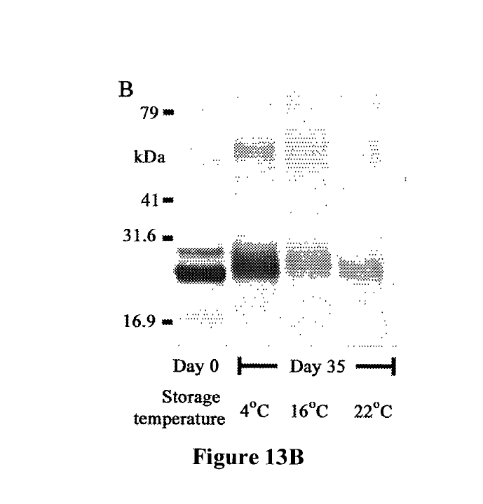

Initially, the effect of higher temperatures was tested. Samples extracted under different test conditions were stored at 4° C. or 16° C. in a constant temperature bath (MicroCooler II, Boekel Scientific) or at room temperature (˜22° C.) in the dark. Samples (˜1 ml volume) were kept in 2 ml screw-cap microcentrifuge tubes (with rubber O-rings to provide a tight seal). Samples were inverted several times and centrifuged for 3 minutes at 10,000 rpm prior to sampling. The presence or absence of pelleted cell debris during storage did not influence the results. For soybean-derived antigen, a reduction in both PAb- and MAb-HBsAg occurred with storage at the higher temperatures tested, which correlated with antigen precipitation from solution, as evidenced by Western blot (FIGS. 13A-B). The higher temperatures also accelerated total soluble protein precipitation (FIG. 13A). To test the effect of sample pH on in-vitro assembly, cell samples were extracted and incubated in carbonate buffer (pH 11) or the standard PBS buffer (pH 7.4).

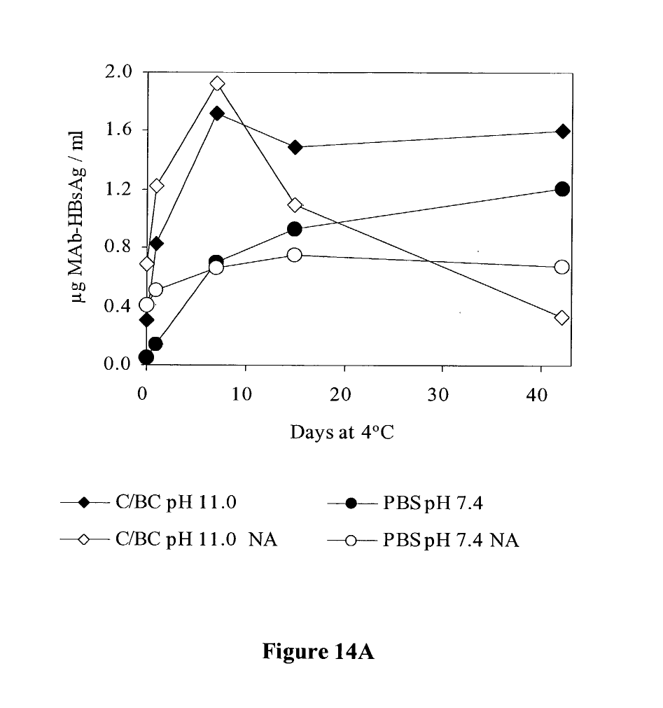

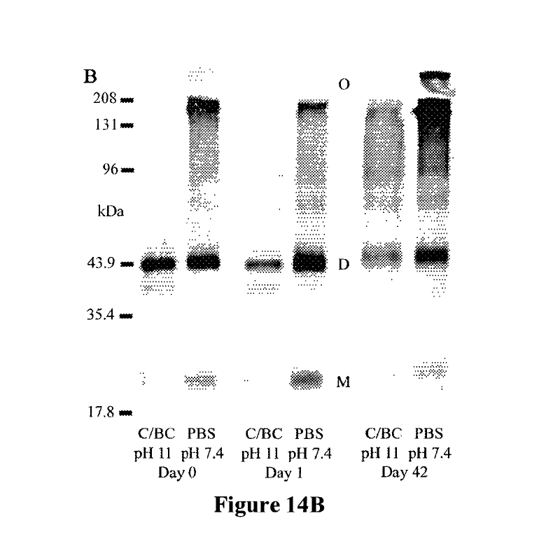

In the absence of sodium ascorbate, MAb-HBsAg formed rapidly at pH 11, plateauing at day 7, at which point it constituted 80% of the initial antigen detectable by the PAb immunoassay (FIG. 14A and Table 4). At pH 7.4, the rise in antigenicity was more gradual and, by day 42, the percentage of MAb- to PAb-HBsAg had reached 59%. When select samples were analyzed by non-reducing Western blot, in vitro disulfide bonding was evident, resulting in higher order oligomers retained by the stacking gel in the PBS-extracted samples after 42 days (FIG. 14B).

TABLE 4
Mab-HBsAg as a percentage of initial PAB-HBsAg*-MAb,
monoclonal antibody reactive; PAb, Polyclonal antibody reactive.

| | Day | | | | |
|---|---|---|---|---|---|
| | 0 | 1 | 7 | 15 | 42 |
| C/BC pH 11.0 | 14% | 38% | 80% | 69% | 75% |
| PBS pH 7.4 | 3% | 7% | 34% | 45% | 59% |

*Transgenic soybean cell extracts. Initial PAb-HBsAg = 2.1 +/− 0.6 μg/mL.

For the carbonate buffer samples, there was a marked absence of higher order oligomers in the initial samples. While cross-linking did occur with storage in the carbonate buffer, the extent of intermolecular disulfide bonding was less than for the PBS-extracted samples. When sodium ascorbate was introduced, the initial MAb-HBsAg profile was similar for the carbonate buffer lacking the antioxidant, with maximum titers attained by day 7; however, levels fell rapidly thereafter.

With the PBS buffer, the profile was similar to that previously observed (FIGS. 11 and 12), the presence of antioxidant reducing the final MAb HBsAg titers (Day 42) by 44%. The initial PAb-HBsAg titers for all four buffer conditions tested was very similar and, in contrast to MAb-HBsAg, either remained constant or dropped only slightly over the 42-day period of study (similar to profiles observed in FIGS. 6 and 7, R=0.6 data).

The fraction of the antigen that displayed the MAb-reactive epitopes was influenced by the buffer pH, storage time at 4° C., and the presence of sodium ascorbate. In vitro assembly of the antigen also occurred yielding higher order oligomers, a fraction of which were retained in the stacking gel (FIGS. 12 and 14). In initial experiments, the titers of MAb-reactive epitopes appeared to correlate with oligomer formation (FIG. 12), suggesting the latter was responsible for the former. However, when carbonate buffer was employed, a doubling of MAb-HBsAg titers occurred after 1 day (FIG. 14A), while no change occurred in the extent of intermolecular disulfide bonding (FIG. 14B). This indicated that an alternative mechanism was responsible for the formation of these epitopes. It has been suggested that the p24^{s}-dimer unit was sufficient for the generation of all the immunogenetically relevant epitopes of HBsAg (Mishiro, et al., “A 49,000-Dalton Polypeptide Bearing All Antigenic Determinants and Full Immunogenicity of 22-nm Hepatitis B Surface Antigen Particles,” J. Immunol. 124:1589-1593 (1980), which is hereby incorporated by reference in its entirety). The virus neutralizing RF1 monoclonal epitope has been shown to be present on dimers but not monomers (Hauser, et al., “Induction of Neutralizing Antibodies in Chimpanzees and in Humans by a Recombinant Yeast-Derived Hepatitis B Surface Particle,” A. J. Zuckerman, Ed. In Viral Hepatitis and Liver Disease, New York, N.Y.: Alan R. Liss, pp. 1031-1037 (1988); Waters, et al., “Virus Neutralizing Antibodies to Hepatitis B Virus: The Nature of an Immunogenic Epitope On The S Gene Peptide,” J. Gen. Virol. 67:2467-2473 (1986), which are hereby incorporated by reference in their entirety), yet interestingly the RF1 monoclonal antibody also binds effectively to a cyclic (i.e. disulfide bonded) synthetic peptide from the antigenic portion of p24^{s} (Waters, et al., “Analysis of the Antigenic Epitopes of Hepatitis B Surface Antigen Involved in the Induction of a Protective Antibody Response,” Virus Res. 22:1-12 (1991), which is hereby incorporated by reference in its entirety). This suggests that dimerization introduces conformational rearrangements of the individual monomers which are prerequisites for intramolecular disulfide bridges. It is these disulfide bridges that ultimately generate certain immunogenic epitopes, such as those recognized by the Auszyme MAb assay.

### Example 10

### Effect of Detergent on Conversion of the Complex High MW HBsAg Found Within Plant Cells to HBsAg VLPs

The production of edible vaccines in transgenic plants and plant cell culture may be improved through a better understanding of antigen processing and assembly. For soybean cells expressing HBsAg, sucrose gradient analysis of crude extracts showed that HBsAg had a complex size distribution uncharacteristic of the antigen's normal structure of uniform 22 nm virus-like particles. Manipulating the detergent to plant material ratio (R) yielded a crude cell extract where the majority of the HBsAg was in the virus-like particle (“VLP”) form. Data presented herein describes how detergent can effect the conversion of the complex high MW HBsAg found within plant cells to HBsAg VLPs more closely resembling the yeast-derived vaccine.

Sucrose Gradients (Method); Sucrose gradients of crude plant-cell extracts were performed on a sucrose step gradient as previously described Mason, et al., “Expression of Hepatitis B Surface Antigen in Transgenic Plants,” Proc. Nat'l. Acad. Sci. USA, 89: 11745-49 (1992), which is hereby incorporated by reference in its entirety. The final gradient concentration was increased from 30% to 50% sucrose to resolve the material that pelleted at the lower sucrose concentration. Samples were centrifuged in a Beckman SW41Ti at 33,000 rpm for 7 hours at 5° C. and 1 mL fractions taken. These fractions were assayed by polyclonal ELISA, the Auszyme monoclonal kit, and Western blot under both reducing and non-reducing conditions. The sucrose concentration of the gradient fractions was determined by refractometry.

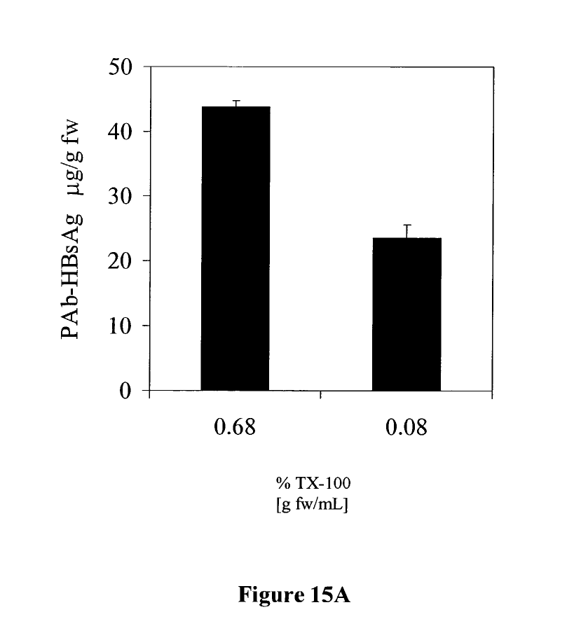

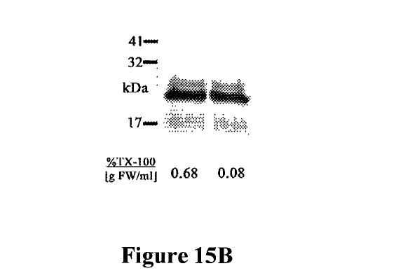

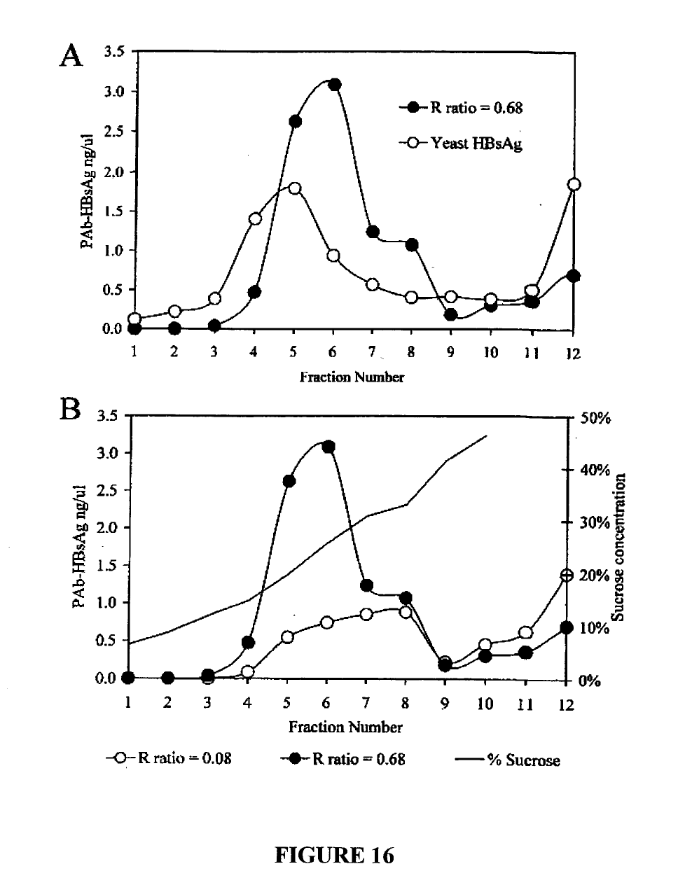

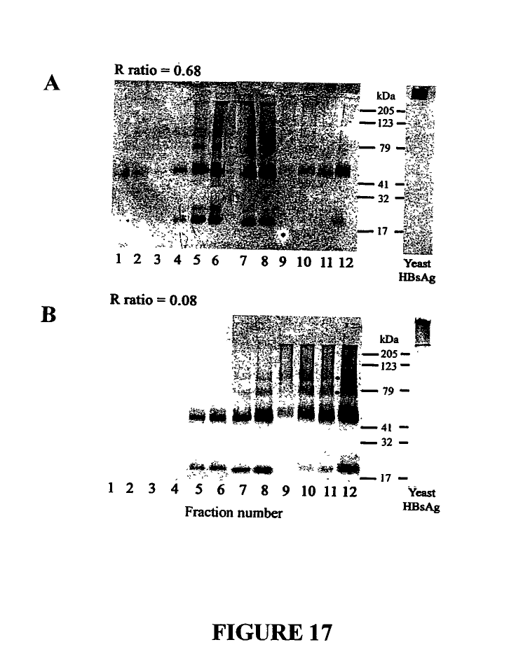

Sucrose Gradient Analysis of Crude Plant Cell Extracts (Results); To better understand the level of membrane association and intracellular form of HBsAg, sucrose gradients of crude soybean cell extracts from were performed. Samples were extracted using two different detergent conditions: a ratio (R) of Triton X-100 concentration (% v/v) to final cell concentration (% TX-100/[g cell fw per mL]) of 0.68 or 0.08. In both cases, the total p24^{s} extracted was identical; however, the level of PAb-HBsAg detectable differed by a factor of 2 (FIG. 15A). These PAb-HBsAg differences were reflected in the sucrose gradient profile: at the higher detergent concentration, a clearly defined peak was observed which sedimented slightly faster than the yeast-derived HBsAg standard (FIG. 16). This profile was consistent with that obtained for HBsAg from transgenic tobacco leaves Mason, et al., “Expression of Hepatitis B Surface Antigen in Transgenic Plants,” Proc. Nat'l. Acad. Sci. USA, 89:11745-49 (1992), which is hereby incorporated by reference in its entirety. In contrast at the lower detergent level, no sharp peak was evident and the proportion of very high molecular weight (VHMW) PAb-reactive material (fractions 9-12) increased by 42% (˜470 ng) (FIG. 16). This increase did not account for the 63% reduction (˜5400 ng) in the peak fractions (4-8). Comparison of the corresponding Western blots, which detect all p24^{s}, as shown in FIG. 17, the vast majority of p24^{s} failed to react with the PAb-immunoassay sedimented at VHMW (compare fractions 9-12 on both blots). This indicates that the peak seen at the higher detergent level (standard extraction buffer) was formed during extraction by detergent-mediated disassociation of VHMW material. Only a small fraction of the intracellular HBsAg was in a particle form which co-migrated with the yeast standard. For tobacco HB155-18 cells and potato tuber extracts, the PAb-HBsAg profiles, under low detergent conditions, were similarly shifted to a higher density relative to the yeast standard.

Sucrose Gradient Analysis of Crude Plant Cell Extracts (Discussion); There are mixed reports regarding the intracellular form of HBsAg; for yeast the only report with visual confirmation by electron microscopy suggests the antigen accumulated as 20-30 nm VLPs (Kitano, et al., “Recombinant Hepatitis B Virus Surface Antigen Accumulates as Particles in Saccharomyces cerevisiae,” Bio/Technology S:281-83 (1987), which is hereby incorporated by reference in its entirety). This is supported by sucrose gradient analysis of detergent treated cell extracts, where the antigen migrated as a single well defined peak (Wampler, et al., “Multiple Chemical Forms of the Hepatitis B Surface Antigen Produced in Yeast,” Proc. Nat'l. Acad. Sci. USA 82:6830-34 (1985), which is hereby incorporated by reference in its entirety). However, electron microscopy of recombinant Chinese Hamster Ovary (CHO) cells showed accumulation of HBsAg as bundles of long filaments (Patzer, et al., “Intracellular Assembly and Packaging of Hepatitis B Surface Antigen Particles Occur in the Endoplasmic Reticulum,” S. Virol. 63: 73-81 (1986), which is hereby incorporated by reference in its entirety). In the presence of detergent, sucrose gradients of soybean cell extracts showed PAb-HBsAg migrating as a well defined peak (FIG. 16) and the total p24^{s} profile was similar (FIG. 17). This indicated the presence of a relatively uniform VLP population, having a marginally higher MW than the yeast-standard. However, when detergent concentration was reduced 8-fold, the VLP-peak disappeared and PAb-HBsAg was halved, with the undetectable portion of the antigen migrating at very high molecular weight (FIGS. 15-17). The marked lack of PAb-HBsAg co-migrating with the yeast-derived antigen indicates that VLPs are not the predominant intracellular form of HBsAg in plant cells, but they can be formed upon extraction in the presence of detergent. For plant cell extracts, the very high molecular weight unreactive material most probably represents tracts of ER membrane, where the antigenically reactive regions of p24^{s} are on the lumenal membrane face and therefore shielded.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A composition comprising a hepatitis B surface antigen stabilized with a milk protein and/or a milk protein component.

2. A composition according to claim 1, wherein the hepatitis B surface antigen comprises hepatitis B surface antigen protein in dimer and higher multimer forms of membrane associated disulfide cross-linked small surface antigen proteins (p24^{s}).

3. A composition according to claim 2, wherein the dimer and higher multimer forms are purified serum-derived HBsAg antigens.

4. A composition according to claim 1, wherein the hepatitis B surface antigen is stabilized with a milk protein selected from the group consisting of soy milk protein, skim milk protein, and mixtures thereof.

5. A composition according to claim 1, wherein the hepatitis B surface antigen is stabilized with a milk protein component selected from the group consisting of lactose, casein or sodium caseinate, whey protein, minerals, and mixtures thereof.

6. A composition according to claim 1, wherein the composition further comprises a buffer solution into which the milk protein and/or the milk protein component is incorporated.

7. A composition according to claim 6, wherein the milk protein and/or the milk protein component are present in the buffer solution at a level of 5% to 15% by weight volume (w/v).

8. A composition according to claim 6, wherein the buffer solution further includes an antioxidant.

9. A composition according to claim 8, wherein the antioxidant is selected from the group consisting of sodium ascorbate, sodium metabisulphite, and mixtures thereof.

10. A composition according to claim 8, wherein the anti-oxidant is present in the buffer solution at a level of up to 20% by weight per volume (w/v).

11. A composition according to claim 10, wherein the anti-oxidant is present in the buffer solution at a level of 1.5% to 2% by weight per volume (w/v).

12. A composition according to claim 1, wherein the milk protein and/or the milk protein component is in powder form or in liquid form.

13. A composition according to claim 1, wherein the composition is in solubilized form or in dried solid form.

14. An oral vaccine for treatment of hepatitis B comprising:
the composition of claim 1 and
a carrier.

15. A method of immunizing a subject against hepatitis comprising:
administering to the subject the stabilized composition of hepatitis B surface antigen according to claim 1.

16. A method according to claim 15, wherein said administering is carried out orally.

17. A method according to claim 16, wherein said administering is carried out orally in a form selected from the group consisting of an oral vaccine, a controlled release preparation, and a sublingually administered preparation.

18. A method according to claim 17, wherein the stabilized composition of hepatitis B surface antigen is administered as an oral vaccine.

19. A method according to claim 18, wherein the oral vaccine is in a form selected from the group consisting of a capsule, a tablet, a microsphere, an encapsulated microsphere, and a suspension.

20. A method of producing a stabilized hepatitis B surface antigen protein comprising:
providing a cell culture suspension containing hepatitis B surface antigen and
extracting said hepatitis B surface antigen with a milk protein and/or a milk protein component to yield a stabilized hepatitis B surface antigen protein extract.

21. A method according to claim 20, wherein the hepatitis B surface antigen is stabilized with a milk protein selected from the group consisting of skim milk and soy milk.

22. A method according to claim 21, wherein the hepatitis B surface antigen is stabilized with a milk protein component selected from the group consisting of lactose, casein, sodium caseinate, whey protein, and minerals.

23. A method according to claim 20, wherein said providing a cell culture suspension is achieved by transforming a plant cell culture suspension with a nucleic acid encoding a hepatitis B surface antigen.

24. A method according to claim 23, wherein said transforming a plant cell culture suspension is bacterially mediated.

25. A method according to claim 24, wherein the bacteria is Agrobacterium tumefaciens.

26. A method according to claim 23, wherein said transforming is carried out by particle bombardment.

27. A method according to claim 23, wherein the plant cellular culture suspension is derived from plants or plant extracts selected from the group consisting of tobacco, soy bean, tuber, mustard plant, tomato, alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, sorghum, sugarcane, and Arabidopsis thaliana.

28. A method according to claim 23, wherein the plant cell culture suspension is derived from or selected from the group consisting of stationary phase cultures of lines of tobacco NT1 (nicotiana tobacum) cell suspension cultures (line NT1 HB155-18), soy bean (glycine max) cell suspension cultures (line W82 HB155-37).

29. A method of claim 23, wherein said extracting is carried out with detergent at a ratio of final detergent concentration (% v/v) to plant material concentration (grams of fresh weight per ml) of 0.4 to 0.6.

30. The method according to claim 20, further comprising:
separating non-hepatitis B surface antigen protein suspension extracts from the stabilized hepatitis B surface antigen protein extract.

31. The method according to claim 20, wherein said providing is carried out by maintaining the hepatitis B surface antigen containing cell culture extract at a stable temperature range of about 4° C. to about 25° C.

32. A method according to claim 20, wherein said extracting is carried out with an extraction buffer comprising an antioxidant.

33. A method according to claim 32, wherein the extraction buffer includes the milk protein and/or the milk protein component.

34. A method according to claim 32, wherein the hepatitis B surface antigen is stabilized with the milk protein and/or the milk protein component which are present in the extraction buffer at a level of 5% to 15% by weight volume (w/v).

35. A method according to claim 32, wherein the extraction buffer is maintained at a pH range of 6 to 8.

36. A method according to claim 32, wherein the antioxidant is selected from the group consisting of sodium ascorbate, sodium metabisulphite, and mixtures thereof.

37. A method according to claim 32, wherein the antioxidant is present in the buffer solution at a level of up to 20% by weight per volume (w/v).

38. A method according to claim 37, wherein the antioxidant is present in the buffer solution at a level of 1.5% to 2% by weight per volume (w/v).

39. A method of increasing immunogenicity of a hepatitis B surface antigen, said method comprising:
providing a cell culture medium comprising a hepatitis B surface antigen;
extracting the hepatitis B surface antigen with a buffer containing a pH of 7 to 12 to yield an extract; and
storing the extract at 0 to 10° C. so that the hepatitis B surface antigen has an increased immunogenicity.

40. A method according to claim 38, wherein the buffer contains a detergent.
